SEQUENCE LISTING (1 sequence)

SEQ ID NO: 1 - DNA, 13 bp, synthetic construct
  source 1..13: mol_type = other DNA
attgcttgcttcg

(19) United States
(12) Patent Application Publication
(10) Pub. No.: US 2026/0057967 A1
(43) Pub. Date: Feb. 26, 2026

(54) METHOD FOR PREDICTING THE MELTING TEMPERATURE OF OLIGONUCLEOTIDE

(71) Applicant: SEEGENE, INC., Seoul (KR)

(72) Inventors: Mi Hyun JANG, Seoul (KR); Han Bit LEE, Seoul (KR); Young Jo LEE, Seoul (KR)

(21) Appl. No.: 19/279,622

(22) Filed: Jul. 24, 2025

Related U.S. Application Data
(63) Continuation of application No. 17/050,030, now Pat. No. 12,394,503.

(30) Foreign Application Priority Data
Jun. 29, 2018 (KR) 10-2018-0075939

(51) Int. Cl. G16B 40/00

(52) U.S. Cl. CPC G16B 40/00; C12Q 2527/107

(57) ABSTRACT

The present invention relates to a method for predicting the melting temperature (Tₘ) of an oligonucleotide, in particular a primer or probe, in a PCR or hybridization assay. The method of present invention can accurately predict the Tₘ of an oligonucleotide in various reaction environments using the equations for Tₘ calculation, the equation including parameter values optimized for the reaction environment in which the oligonucleotide is to be used.

### CROSS-REFERENCE TO RELATED APPLICATIONS

This application is a continuation of U.S. patent application Ser. No. 17/050,030, filed on Oct. 23, 2020, which is a national phase application of PCT Application No. PCT/KR2019/007908, filed on Jun. 28, 2019, which claims priority to Korean Patent Application No. 10-2018-0075939, filed on Jun. 29, 2018. The entire disclosures of the applications identified in this paragraph are incorporated herein by references.

### SEQUENCE LISTING

This application contains references to amino acid sequences and/or nucleic acid sequences which have been submitted concurrently herewith as the sequence listing XML file entitled “000231uscoa_SequenceListing”, file size 1,961 bytes, created on 11 Nov. 2025. The aforementioned sequence listing is hereby incorporated by reference in its entirety pursuant to 37 C.F.R. § 1.52(e) (5).

### FIELD

The present invention relates to a method for predicting the melting temperature of an oligonucleotide, particularly a primer or probe in a PCR or hybridization assay.

### BACKGROUND

Hybridization between complementary nucleic acids is one of the important features in the Watson-Crick Model for DNA structure and is used in many biological and biomedical fields. For example, a method of replicating or amplifying a nucleic acid molecule is initiated by the step of hybridizing a complementary oligonucleotide (typically, a primer) to a specific site of a target nucleic acid molecule. Then, a polymerase uses the target nucleic acid molecule as a template to synthesize a complementary nucleic acid from the primer.

A method known as polymerase chain reaction (PCR) has been widely used in the biological and biomedical fields. In PCR, two or more primers that hybridize to different sites of the target nucleic acid are used. The target nucleic acid sequence and its complement are then amplified through multiple cycles of heating and cooling to repeatedly hybridize and dissociate complementary strands. The amplification allows a small amount of target nucleic acid to be used for detection, sequencing, and the like.

Multiplex PCR is a modification of PCR that uses multiple pairs of primers in a single PCR mixture to amplify and detect a plurality of nucleic acids in a sample. This technique allows for simultaneous amplification and evaluation of multiple different target nucleic acids in a sample. However, successful multiplex PCR is not attainable due to the difficulty in selection and design of suitable oligonucleotides.

Another technique widely used in the biological and biomedical fields employs nucleic acid hybridization to detect a target nucleic acid sequence in a sample. Such technique, for example, Southern blotting and the like, immobilizes target nucleic acid molecules in a sample on a solid surface or support (e.g., a membrane support). The immobilized target nucleic acid molecule is hybridized with one or more complementary nucleic acids (referred to as probes) and detected by measuring a signal from a detectable label on the probe.

The success of such techniques including nucleic acid hybridization depends on the use of primers and probes that specifically hybridize to the target nucleic acid molecule. This is particularly critical in multiplex PCR involving more oligonucleotides.

The stability of oligonucleotides such as primers or probes is often expressed as the melting temperature (Tₘ) of the duplex between an oligonucleotide and its complementary strand. The Tₘ is defined as the temperature at which half of the duplex will dissociate to become single-stranded. Preferably, the nucleic acid hybridization is carried out at a temperature slightly lower than Tₘ to optimize hybridization between the primer or probe and its target nucleic acid and minimize non-specific hybridization of the primer or probe to the non-target nucleic acid. The Tₘ is also important in PCR involving thermal cycling.

Conventionally, various methods for predicting the Tₘ of a specific oligonucleotide have been proposed.

Marmur and Doty (1962) used a rather simple formula where the Tₘ depends only on the relative content of cytosine and guanine. This formula was later improved by adding a correction factor which is also responsible for salt concentration, thus adjusting the Tₘ value for different experimental conditions (Wetmur, 1991). An indepth analysis of DNA oligonucleotides and their corresponding experimental Tₘs has led to the conclusion that not only the relative amounts of cytosine and guanine concentrations determine the thermal denaturation of DNA, but also the sequential arrangement of different nucleotides in DNA sequences were found to play a major role in the experimental value of Tₘs. Later, Breslauer et al. (Proc. Natl. Acad. Sci. USA 1986, 83: 3746-3750) adopted a Tₘ prediction model known as the “nearest-neighbor (NN)” model (SantaLucia et al., Biochemistry 1996, 35: 3555-3562; Santa Lucia, Proc Natl Acad Sci USA 1998, 95: 1460-1465). The NN model uses nearest-neighbor thermodynamic parameters and several optimized NN tables with NN parameters have been published (Gotoh and Tagashira, 1981; Vologodskii et al., 1984; Breslauer et al., 1986; Delcourt and Blake, 1991; Doktycz et al., 1992; SantaLucia et al., 1996; Sugimoto et al., 1996; Allawi and Santa Lucia, 1997).

The NN models use the values of the NN parameters obtained under a fixed reaction environment (e.g., 1M NaCl). However, the reaction environment in which oligonucleotides for Tₘ prediction are tested may vary widely. Therefore, the use of the NN parameter values optimized for a fixed reaction environment may lead to inaccurate prediction of the Tₘs of oligonucleotides, because such values do not accurately reflect all the various reaction environments.

In order to address the issue, there has been proposed a method of using a certain correction factor (such as a correction factor related to salt concentration) in addition to the NN parameters. However, since the Tₘ of the oligonucleotide is also affected by many other factors and it is difficult to establish the formula for Tₘ calculation considering all of these correction factors, the approach using a correction factor is also not suitable for predicting an accurate Tₘ that reflects the entire reaction environment. Further, the above method assumes that the values of the NN parameters for each NN sequence (also referred to as NN propagation) do not change for all reaction environments, except for the value of the correction factor. However, since such changes in the reaction environment also affect the values of the thermodynamic NN parameters for each NN sequence, the above approach has limitations in accurately predicting the Tₘ of the oligonucleotide in various reaction environments.

Therefore, there is still a need to develop a novel method for accurately predicting the Tₘ of an oligonucleotide in a variety of reaction environments.

Throughout this application, various patents and publications are referenced and citations are provided in parentheses. The disclosure of these patents and publications in their entirety are hereby incorporated by references into this application in order to more fully describe this invention and the state of the art to which this invention pertains.

### SUMMARY

The present inventors have endeavored to develop a method for accurately predicting the melting temperature (Tₘ) of an oligonucleotide of interest in various reaction environments. As a result, the present inventors have established an equation for Tₘ calculation for each of the differently defined reaction environments, using a plurality of reference data sets, each comprising information about the sequences of a plurality of reference oligonucleotides and information about the experimental Tₘs of the plurality of reference oligonucleotides in a defined reaction environment, and have confirmed that the use of the equation for Tₘ calculation allows for accurate prediction of the Tₘ of an oligonucleotide of interest in various reaction environments.

Accordingly, it is an object of the present invention to provide a method for predicting the Tₘ of an oligonucleotide.

It is another object of the present invention to provide a computer readable storage medium containing instructions to configure a processor to perform a method for predicting the Tₘ of an oligonucleotide.

It is still another object of the present invention to provide an apparatus for predicting the Tₘ of an oligonucleotide.

Other objects and advantages of the present invention will become apparent from the detailed description to follow taken in conjugation with the appended claims and drawings.

### BRIEF DESCRIPTION OF THE DRAWINGS

FIG. 1 shows a flow diagram illustrating a method in accordance with an embodiment of the present invention.

FIG. 2 shows (A) a scatter plot illustrating the correlation between the experimental Tₘ and the predicted Tₘ calculated using the conventional known NN parameters, and (B) a scatter plot illustrating the correlation between the experimental Tₘ and the predicted Tₘ calculated using the NN parameters of the present invention.

FIG. 3A shows (A) a scatter plot illustrating the correlation between the experimental Tₘ and the predicted Tₘ calculated using Equation I-1 and (B) a scatter plot illustrating the correlation between the experimental Tₘ and the predicted Tₘ calculated using Equation III-1.

FIG. 3B shows (A) a scatter plot illustrating the correlation between the experimental Tₘ and the predicted Tₘ calculated using the Equation IV-1 and (B) a scatter plot illustrating the correlation between the experimental Tₘ and the predicted Tₘ calculated using the Equation V-1.

### DETAILED DESCRIPTION

### I. Method for Predicting the Melting Temperature (Tₘ) of an Oligonucleotide in Differently Defined Reaction Environments

In a first aspect of this invention, there is a method for predicting the melting temperature (Tₘ) of an oligonucleotide of interest, comprising the steps of:
(a) providing a plurality of reference data sets, wherein the reference data set comprises (i) information about the sequences of a plurality of reference oligonucleotides and (ii) information about the experimental Tₘs of the plurality of reference oligonucleotides in a defined reaction environment, wherein the plurality of reference data sets are generated in differently defined reaction environments;
(b) establishing an equation for Tₘ calculation for each of the differently defined reaction environments using each of the plurality of reference data sets including the information (i)-(ii); wherein the establishment of the equation comprises determining the values of the parameters in the equation, wherein the parameters comprise nearest-neighbor (NN) thermodynamic parameters; and
(c) calculating the Tₘ of an oligonucleotide of interest in one among the differently defined reaction environments using the equation established for the one among the differently defined reaction environments.

The present inventors have endeavored to develop a method for accurately predicting the melting temperature (Tₘ) of an oligonucleotide of interest in various reaction environments. As a result, the present inventors have established an equation for Tₘ calculation for each of the differently defined reaction environments, using a plurality of reference data sets, each comprising information about the sequences of a plurality of reference oligonucleotides and information about the experimental Tₘs of the plurality of reference oligonucleotides in a defined reaction environment, and have confirmed that the use of the equation for Tₘ calculation allows for accurate prediction of the Tₘ of an oligonucleotide of interest in various reaction environments.

The Tₘ prediction of the present invention is based on a nearest-neighbor (NN) model using thermodynamic nearest-neighbor parameters. Nearest-neighbor model-based Tₘ prediction is well known in the art (see Breslauer et al., Proc. Natl. Acad. Sci. U.S.A. 1986, 83: 3746-3750; Santa Lucia et al., Biochemistry 1996, 35: 3555-3562; Santa Lucia, Proc. Natl. Acad. Sci. U.S.A. 1998, 95: 1460-1465, which are incorporated herein by reference in their entirety).

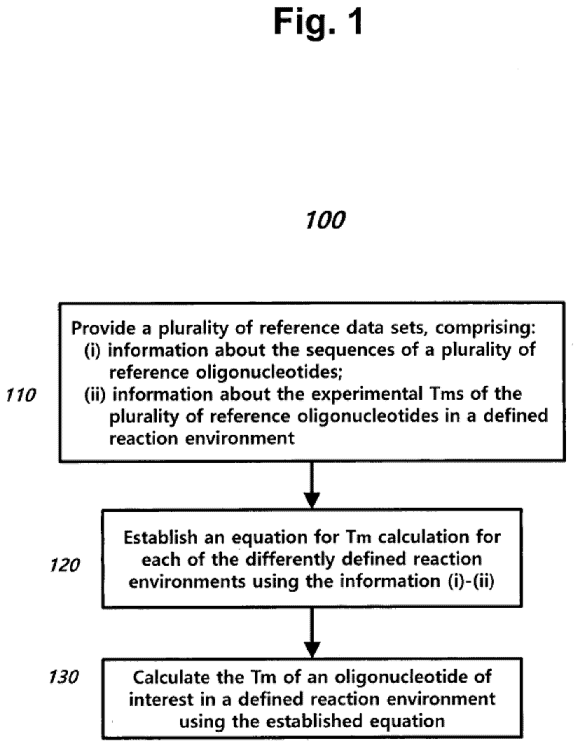

FIG. 1 shows a flow diagram illustrating each step of a method 100 in accordance with an embodiment of the present invention. The method of the present invention will now be described with reference to FIG. 1.

### Step (a): Providing a Plurality of Reference Data Sets 110

First, a plurality of reference data sets is provided 110. The reference data set comprises (i) information about the sequences of a plurality of reference oligonucleotides and (ii) information about the experimental Tₘs of the plurality of reference oligonucleotides in a defined reaction environment, wherein the plurality of reference data sets are generated in differently defined reaction environments.

The term “oligonucleotide” as used herein refers to a linear oligomer of natural or modified monomers or linkages, including deoxyribonucleotides, ribonucleotides and the like, capable of specifically hybridizing with a target nucleotide sequence, whether occurring naturally or produced synthetically. The oligonucleotide is preferably single stranded for maximum efficiency in hybridization. Preferably, the oligonucleotide is an oligodeoxyribonucleotide. The oligonucleotide of this invention can be comprised of naturally occurring dNMP (i.e., dAMP, dGM, dCMP and dTMP), nucleotide analogs, or nucleotide derivatives. The oligonucleotide can also include ribonucleotides. For example, the oligonucleotide of this invention may include nucleotides with backbone modifications such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

The oligonucleotide as used herein is generally less than 200 nucleotides, particularly less than 150 nucleotides, more particularly less than 100 nucleotides, still more particularly less than 50 nucleotides and most particularly less than 30 nucleotides in length.

The oligonucleotides as used herein can be classified into two categories: (i) oligonucleotide whose Tₘ is to be predicted; (ii) oligonucleotide which is used to determine the parameter values included in an equation for Tₘ calculation in a defined reaction environment.

As used herein, the term “oligonucleotide whose Tₘ is to be predicted” refers to an oligonucleotide having no experimental Tₘ, i.e., an oligonucleotide whose Tₘ is unknown. The oligonucleotide is one whose Tₘ information is required for an optimized reaction in PCR, hybridization assay and the like. The oligonucleotide may be referred to herein as “oligonucleotide of interest”, or “unknown oligonucleotide”.

On the other hand, the term “oligonucleotide used in the calculation of the parameter values included in an equation for Tₘ calculation” refers to an oligonucleotide having an experimental (experimentally determined) Tₘ in a defined reaction environment. The oligonucleotide serves to provide information about its sequence as well as information about its Tₘ to determine the parameter values in a defined reaction environment. The oligonucleotide may be referred to herein as “reference oligonucleotide”, “learning oligonucleotide” or “benchmark oligonucleotide”.

As used herein, the term “reference data set” refers to a data set that contains information used to establish a nearest-neighbor (NN) model, specifically an equation for Tₘ calculation, optimized for a defined reaction environment. The reference dataset is used to establish an NN model in a defined reaction environment and then to calculate the Tₘ of an oligonucleotide of interest in the defined reaction environment.

As used herein, the term “a plurality of reference data sets” means at least two reference data sets. In one embodiment, a plurality of reference data sets comprises 2, 3, 4, 5, 6, 7, 8, 9, 10, or more reference data sets.

The reference data set as provided in this step is generated in a defined reaction environment. The reference data set as provided in this step may be generated by preparing a plurality of reference oligonucleotides of varying lengths and sequences and determining their experimental Tₘs.

The plurality of reference data sets are generated in differently defined reaction environments. For example, a first reference data set is generated in a first defined reaction environment and a second reference data set is generated in a second defined reaction environment; wherein the first defined reaction environment is different from the second defined reaction environment.

The reference data set corresponds to a defined reaction environment, and the plurality of reference data sets corresponds to differently defined reaction environments.

In other words, each reference data set is used herein to establish an NN model in each reaction environment and to calculate the Tₘ of an oligonucleotide of interest in each reaction environment, and the plurality of reference data sets is used to establish several NN models in differently defined reaction environments and to calculate the Tₘ of an oligonucleotide of interest in the differently defined reaction environments.

The number of reference data sets corresponds to the number of the differently defined reaction environments. For example, three reference data sets correspond to three differently defined reaction environments.

Each of the reference data sets herein may be referred to as a first reference data set, a second reference data set, or the like, for identification. In this case, the first reference data set is provided to establish an equation for Tₘ calculation having the parameter values optimized for a first defined reaction environment, and the second reference data set is provided to establish an equation for Tₘ calculation having the parameter values optimized for a second defined reaction environment.

The reference data set comprises (i) information about the sequences of a plurality of reference oligonucleotides and (ii) information about the experimental Tₘs of the plurality of reference oligonucleotides in a defined reaction environment. The reference data set may further comprise other information, and the information may be used to establish an equation for Tₘ calculation. The components included in the reference data set will be described in detail.

### (i) Information About the Sequences of a Plurality of Reference Oligonucleotides

Each reference data set includes information about the sequences of a plurality of reference oligonucleotides. The term “information about the sequences of a plurality of reference oligonucleotides” means information derived from the nucleic acid sequences of a plurality of reference oligonucleotide molecules.

In one embodiment, the information about the sequences of a plurality of reference oligonucleotides comprises linear orders of a plurality of reference oligonucleotides.

In one embodiment, the information about the sequences of a plurality of reference oligonucleotides comprises nearest-neighbor (NN) sequences derived from a plurality of reference oligonucleotides.

In one embodiment, the information about the sequences of a plurality of reference oligonucleotides comprises the frequency for each of nearest-neighbor (NN) sequences derived from a plurality of reference oligonucleotides.

In one embodiment, the information about the sequences of a plurality of reference oligonucleotides comprises the length of a plurality of reference oligonucleotides.

In one embodiment, the information about the sequences of a plurality of reference oligonucleotides comprises the content of A, T (U), G, C, or a combination thereof in a plurality of reference oligonucleotides.

The number of the sequences of the reference oligonucleotides included in each reference data set is not particularly limited, as long as all the values of the thermodynamic parameters for the nearest-neighbor (NN) sequences (also referred to as NN propagation, preferably a total of 10 NN sequences) can be determined using the sequences of the reference oligonucleotides. The number of the sequences of the reference oligonucleotides included in each reference data set is preferably as large as possible for accurate determination of the NN parameter values, but it may be adjusted to reduce the time required for determination of the NN parameter values, and the like. For example, the number of the sequences of the reference oligonucleotides in each reference data set may be at least 20, at least 30, or at least 50, up to 100, up to 300, up to 500, up to 1000, up to 3000, or 5000.

The sequences of the reference oligonucleotides constituting each reference data set may be the same or different for each reference data set.

In one embodiment, the sequences of the reference oligonucleotides in the first reference data set is totally identical to those in the second reference data set (the reaction environment in which the Tₘs of the reference oligonucleotides in the first reference data set are measured is different from that in which the Tₘs of the reference oligonucleotides in the second reference data set is measured).

In another embodiment, the sequences of the reference oligonucleotides in the first reference data set is partially identical to those in the second reference data set (the reaction environment in which the Tₘs of the reference oligonucleotides in the first reference data set are measured is different from that in which the Tₘs of the reference oligonucleotides in the second reference data set is measured).

In still another embodiment, the sequences of the reference oligonucleotides in the first reference data set is totally different from those in the second reference data set (the reaction environment in which the Tₘs of the reference oligonucleotides in the first reference data set are measured is different from that in which the Tₘs of the reference oligonucleotides in the second reference data set is measured).

The sequences of a plurality of reference oligonucleotides in one reference data set may be used to construct another reference data set.

### (ii) Information About the Experimental Tₘs of the Plurality of Reference Oligonucleotides in a Defined Reaction Environment

Each reference data set includes information about the experimental Tₘs of the plurality of reference oligonucleotides in a defined reaction environment. Since each reference data set includes not only sequences of reference oligonucleotides but also their experimental Tₘs as components, the identity between two reference data sets is determined in consideration of the two components.

As used herein, the term “experimental melting temperatures of a plurality of reference oligonucleotides in a defined reaction environment” refers to an experimental measure of melting temperature of each of the reference oligonucleotides in a defined reaction environment.

As used herein, the term “reaction environment” refers to one in which the oligonucleotide whose Tₘ is to be predicted (oligonucleotide of interest) is used/placed/applied. In other words, the term “reaction environment” refers to the medium, particularly chemical characteristics, surrounding an oligonucleotide. The reaction environment may be defined by the types and concentrations of the components used in a reaction except the oligonucleotide of interest. For example, when an oligonucleotide whose Tₘ is to be predicted is used in a PCR reaction, examples of the components can be used in the reaction include, without limitation, a buffers (for pH adjustment) such as Tris, Tricine, bis-Tricine, HEPES, EPPS, CHES and CAPSO; monovalent ions such as KCl, (NH₄)₂SO₄, NaCl, LiCl, NH₄Cl, NaN₃, CoCl₂, KOAc, KSO4, (Mn(OAc)₂ and NaOAc; divalent ions such as MgCl₂, MgSO₄, MgSO₄, and ZnCl₂; stabilizers such as BSA and gelatin; proteins such as T4 gene 32 protein and Et SSB; non-ionic detergents such as NP-40, Tween20, Triton X-100, Triton X-114, CHAPSO, IGEPAL, CHAPS, MEGA-8, Brij 35 and BRIJ 58; reducing agents such as DTT, b-mercaptoethanol and TCEP; compatible solutes such as glycerol, trehalose, sucrose, xylitol, melezitose, xylene cyanol, mannose, sorbitol, mannitol, betaine, ectoine and proline; sulfoxides such as DMSO; amides such as formamide; tetraalkyl-ammonium salts such as TMAC and TMAA; polyhydroxyl alcohols such as propylene glycol and polyvinyl alcohol (PVA); chelating agents such as EDTA and EGTA; enzymes such as polymerases, dNTPs, and the like.

The plurality of reference data sets are generated in differently defined reaction environments.

The term of “differently defined reaction environments” means that types and/or concentrations of any component used in a reaction except the oligonucleotide of interest is different, the component selected from the group consisting of (a) buffers, (b) monovalent ions; (c) divalent ions; (d) stabilizers; (e) proteins; (f) non-ionic detergents; (g) reducing agents; (h) compatible solutes; (i) sulfoxides; (j) amides; (k) tetraalkyl-ammonium salts; (l) polyhydroxyl alcohols; (m) chelating agents; (n) enzymes; (o) dNTPs; and (p) combinations thereof, and the like.

For example, a first reference data set is generated in a first defined reaction environment, and a second reference data set is generated in a second defined reaction environment; wherein the first defined reaction environment and the second defined reaction environment are different from each other. The difference between the first defined reaction environment and the second defined reaction environment indicates that the composition of the two reaction environments is different. For example, this may include the case where any one component is present in the first defined reaction environment but not in the second defined reaction environment as well as the case where the concentration of any one component in the first defined reaction environment is different from the concentration in the second defined reaction environment.

In other words, the defined reaction environment herein is different for each reference data set. As used herein, the expression “the defined reaction environment is different for each reference data set” means that the reaction environment in which the melting temperatures of reference oligonucleotides in a reference data set are experimentally measured is different from the reaction environment in which the melting temperatures of reference oligonucleotides in another reference data set are experimentally measured. As an example, a first reference data set may include the melting temperatures of reference oligonucleotides, experimentally measured in a reaction environment containing 1 M NaCl, while a second reference data set may include the melting temperatures of reference oligonucleotides, experimentally measured in a reaction environment containing 0.1 M NaCl. As another example, a first reference data set may include the melting temperatures of reference oligonucleotides, experimentally measured in a first reaction environment, while a second reference data set may include the melting temperatures of reference oligonucleotides, experimentally measured in a second reaction environment further comprising an additional component.

The number of differently defined reaction environments corresponds to the number of reference data sets. For example, when there are three differently defined reaction environments, the corresponding reference data sets, i.e., three reference data sets are

[symbol]

The differently defined reaction environment means at least two different reaction environments. In one embodiment, the differently defined reaction environments comprise 2, 3, 4, 5, 6, 7, 8, 9, 10, or more reaction environments.

As used herein, the term “experimental melting temperature (Tₘ)” of a reference oligonucleotide refers to a Tₘ of a reference oligonucleotide which is determined experimentally in a defined reaction environment. The experimental Tₘ may be determined by melting curve analysis known in the art. As an example, the experimental Tₘ of a reference oligonucleotide may be determined by measuring the change in absorbance from the duplex between the oligonucleotide and its complement as a function of temperature. As another example, the experimental Tₘ of a reference oligonucleotide may be determined by measuring the change in fluorescence signal from the duplex between the oligonucleotide and its complement (e.g., by using an intercalating dye) as a function of temperature.

It is well known in the art that the Tₘ of an oligonucleotide vary depending on the reaction environment in which the oligonucleotide is used/placed/applied. For the purpose of accurately predicting the Tₘ of an oligonucleotide of interest in a desired reaction environment, the method of the present invention employs the experimental Tₘs of reference oligonucleotides when placed in the same reaction environment as the oligonucleotide of interest.

Thus, reference oligonucleotides for providing their sequences and Tₘs included in a reference data set may be selected from oligonucleotides having experimental Tₘs measured in the same reaction environment as the reaction environment in which an oligonucleotide of interest to be placed, or may be prepared by experimentally measuring the Tₘs of the reference oligonucleotides in the same reaction environment as the reaction environment in which an oligonucleotide of interest to be placed. For example, if it is desired to predict the Tₘ of an oligonucleotide of interest in a first reaction environment, reference oligonucleotides may be selected from oligonucleotides having experimental Tₘs measured in the first reaction environment, or may be prepared by selecting (synthesizing) any oligonucleotides and then experimentally measuring the Tₘs of the oligonucleotides in the first reaction environment.

In one embodiment, each of the plurality of reference data sets comprises a plurality of reference oligonucleotides such that each NN sequence occurs with the frequency of at least two.

As used herein, the term “nearest-neighbor (NN) sequence” refers to a sequence consisting of two adjacent nucleotides (dinucleotide) in an oligonucleotide. The term may be used interchangeably with “nearest-neighbors”, “nearest-neighbor base-pairs”, or “nearest-neighbor pairs”.

For example, in the case of an oligonucleotide sequence consisting of 13 bases of 5′-ATTGCTTGCTTCG-3′ (SEQ ID NO: 1), the total number of nearest-neighbor (NN) sequences is seven (7), i.e., “AT”, “TT”, “TG”, “GC”, “CT”, “TC” and “CG”. In this case, it is noted that “TT” occurs with the frequency of 3, “TG” with the frequency of 2, “GC” with the frequency of 2, “CT” with the frequency of 2.

The nearest-neighbor (NN) sequences possible are sixteen (16) types (4 bases X 4 bases=16), e.g., “AA”, “AT”, “AG”, “AC”, “TA”, “TT”, “TG”, “TC”, “GA”, “GT”, “GG”, “GC”, “CA”, “CT”, “CG” and “CC”. However, it is known that the dinucleotides “AA”, “CA”, “GT”, “CT”, “GA” and “GG” have the same NN parameter values as “TT”, “TG”, “AC”, “AG”, “TC” and “CC”, respectively. Thus, the total number of nearest-neighbor (NN) sequences having parameter values to be determined is ten (10).

For the reference data set, the frequency for each of the NN sequences refers to the number of NN sequences occurred in reference oligonucleotides of the reference data set. That is, the frequency for each of the NN sequences in each reference data set can be obtained by calculating the sum of the number of each NN sequence from entire reference oligonucleotides. For example, if the reference data set has two reference oligonucleotides such as 5′-ATGT-3′ and 5′-GTTA-3′, the reference data set is said to have the frequency of 1 for AT, 1 for GT, 2 for GT, 1 for TT, and 1 for TA.

In one embodiment, each of the plurality of reference data sets comprises reference oligonucleotides such that such that each NN sequence occurs with the frequency of at least 2, at least 3, at least 4, at least 5, at least 7, at least 10, at least 15, at least 20, at least 30, or at least 40.

In one embodiment, each reference data set comprises a plurality of reference oligonucleotides such that the reference oligonucleotides have Tₘs within a certain temperature range. For example, each reference data set comprises a plurality of reference oligonucleotides such that the reference oligonucleotides have Tₘs between 30-100° C., 40-100° C., 50-100° C., 60-100° C., 30-90° C., 40-90° C., 50-90° C., 60-90° C., 30-80° C., 40-80° C., or 50-80° C.

In one embodiment, each reference data set comprises a plurality of reference oligonucleotides such that the reference oligonucleotides have Tₘs with uniform distribution.

In one embodiment, each reference data set comprises at least one reference oligonucleotide having a Tₘ falling within each temperature interval within a temperature range. The lower limit of the temperature range may be 20, 25, 30, 35, 40, 45, 50, 55, 60, 65, 70° C., or more. The upper limit of the temperature range may be 40, 45, 50, 55, 60, 65, 70, 75, 80, 85, 90, 95, 100° C., or more. The each temperature interval within a temperature range may have an interval of 3, 5, 7, 10, 15, 20, 25, 30, 35 or 40° C. As an example, when each temperature interval has an interval of 5° C., 10° C., 15° C. or therebetween, each reference data set may comprise at least one reference oligonucleotide having a Tₘ falling within the each temperature interval. Specifically, when the temperature range consists of temperature intervals of 30-40° C., 40-50° C., 50-60° C., 60-70° C., and 70-80° C., each reference data set may comprise at least one reference oligonucleotide having an experimental Tₘ of 30-40° C., at least one reference oligonucleotide having an experimental Tₘ of 40-50° C., at least one reference oligonucleotide having an experimental Tₘ of 50-60° C., at least one reference oligonucleotide having an experimental Tₘ of 60-70° C., and at least one reference oligonucleotide having an experimental Tₘ of 70-80° C.

### Step (b): Establishment of an Equation for Tₘ Calculation

Afterwards, an equation for Tₘ calculation for each of the differently defined reaction environments is established using each of the plurality of reference data sets including the information (i)-(ii); wherein the establishment of the equation comprises determining the values of the parameters in the equation, wherein the parameters comprise nearest-neighbor (NN) thermodynamic parameters; and

In this step, each of the plurality of reference data sets is used to establish an equation for Tₘ calculation for each reference data set, i.e., for differently defined reaction environments, by determining the values of the parameters included in the equation.

An equation for Tₘ calculation for each of the differently defined reaction environments will be described in detail below.

### Equation for Tₘ Calculation

The defined equation for Tₘ calculation refers to any equation based on an NN model using thermodynamic parameters. The equation may be one known in the art or a modification thereof. The predicted Tₘ of the oligonucleotide may be varied according to the equation used.

The equation for Tₘ calculation used in the present invention is one comprising nearest-neighbor (NN) thermodynamic parameters, or a modification thereof.

In an embodiment, the equation for Tₘ calculation comprises the enthalpy change (ΔH) and the entropy change (ΔS) for each of nearest-neighbor (NN) sequences.

In a particular embodiment, the equation for Tₘ calculation is represented by the following Equation I.

$$Tm = \frac{Δ H ° \times m}{Δ S °} - n \qquad Equation l$$

wherein Tₘ is the melting temperature of the oligonucleotide; ΔH° is the sum of the enthalpy changes; ΔS° is the sum of the entropy changes; m and n are constants.

In the above equation, ΔH° can be calculated by the sum of each NN parameter ΔH°_{NN}, and ΔS° can be calculated by the sum of each NN parameter ΔS°_{NN}, as shown in the following Equation II.

$$Δ H ° = {\sum }_{NN} ( each Δ H {°}_{NN} ) , Δ S ° = {\sum }_{NN} ( each Δ S {°}_{NN} ) \qquad Equation II$$

In the Equation I, m may be 1000 and n may be 273.15, but the m and n may be adjusted by the user. The equation for Tₘ calculation can be found in SantaLucia, J. Jr (2007) Physical principles and visual-OMP software for optimal PCR design. Methods Mol. Biol., 402, 3-34.

In one specific embodiment, Equation I can also be expressed as Equation I-1.

$$T m = \frac{Δ H ° \times 1000}{Δ S °} - 273.15 \qquad Equation I ‐ 1$$

In an embodiment, the equation for Tₘ calculation comprises the parameters of the enthalpy change (ΔH) and the entropy change (ΔS) for each of nearest-neighbor (NN) sequences, and one or more additional parameters.

In an embodiment, the one or more additional parameters comprise a parameter for correction (or supplement, amendment) of entropy change and/or a parameter for correction (or supplement, amendment) of Tₘ contribution by length of the oligonucleotide.

In a particular embodiment, the equation for Tₘ calculation is represented by the following Equation III.

$$Tm = \frac{Δ H ° \times m}{Δ S ° + α} - n \qquad Equation III$$

wherein Tₘ is the melting temperature of the oligonucleotide; ΔH° is the sum of the enthalpy changes; ΔS° is the sum of the entropy changes; α is a first additional parameter for correction of entropy change; and m and n are constants.

In this equation, the first additional parameter α for correction of entropy change is one determined additionally in step (b) besides the thermodynamic parameters (ΔH° and ΔS°). The first additional parameter may be referred to as “first correction parameter” or “parameter associated with entropy change”. The value of the first additional parameter depends on the reaction environment.

In the above equation, ΔH° and ΔS° can be calculated as described above.

In one embodiment, m is 1000 and n is 273.15. The m and n may be adjusted by the user.

In one specific embodiment, Equation III can also be expressed in Equation III-1.

$$Tm = \frac{Δ H ° \times 1000}{Δ S ° + α} - 273.15 \qquad Equation III ‐ 1$$

In another embodiment, the equation for Tₘ calculation may be represented by the following Equation IV.

$$Tm = \frac{Δ H ° \times m}{Δ S °} + β ln ( length ) - n \qquad Equation IV$$

wherein Tₘ is the melting temperature of the oligonucleotide; ΔH° is the sum of the enthalpy changes; ΔS° is the sum of the entropy changes; β is a second additional parameter for correction of Tₘ contribution by length of the oligonucleotide; length is the length of the oligonucleotide; m and n are constants.

In this equation, the second additional parameter β is one determined additionally in step (b) besides the thermodynamic parameters (ΔH° and ΔS°). The second additional parameter may be referred to as “second correction parameter”; “parameter associated with the length of the oligonucleotide sequence”, or “parameter for the relationship between the length and Tₘ of the oligonucleotide sequence”. The second additional parameter β is one for reflecting the influence of the reaction environment on the length of the oligonucleotide. The value of the second additional parameter depends on the reaction environment.

In the above equation, ΔH° and ΔS° can be calculated as described above.

In one embodiment, m is 1000 and n is 273.15. The m and n may be adjusted by the user.

In one specific implementation, Equation IV can also be expressed by Equation IV-1.

$$Tm = \frac{Δ H ° \times 1000}{Δ S °} + β ln ( length ) - 273.15 \qquad Equation IV ‐ 1$$

In another embodiment, the equation for calculation Tₘ may be represented by the following Equation V.

$$Tm = \frac{Δ H ° \times m}{Δ S ° + α} + β ln ( length ) - n \qquad Equation V$$

wherein Tₘ is the melting temperature of the oligonucleotide; ΔH° is the sum of the enthalpy changes; ΔS° is the sum of the entropy changes; α is a first additional parameter for correction of entropy change; β is a second additional parameter for correction of Tₘ contribution by length of the oligonucleotide; length is the length of the oligonucleotide; and m and n are constants.

In this equation, the first additional parameter and the second additional parameter are those determined additionally in step (b) besides the thermodynamic parameters (ΔH° and ΔS°).

In the above equation, ΔH°, ΔS°, α, and β can be calculated as described above.

In one embodiment, m is 1000 and n is 273.15. The m and n may be adjusted by the user.

In one specific embodiment, Equation V can also be expressed as Equation V-1.

$${Tm}_{( α , β )} = \frac{Δ H ° \times 1000}{Δ S ° + α} + β ln ( length ) - 273.15 \qquad Equation V ‐ 1$$

In this equation, the first additional parameter and the second additional parameter are those determined additionally in step (b) besides the thermodynamic parameters (ΔH° and ΔS°).

The equation for Tₘ calculation may be selected by one of skill in the art, and it should be understood that various equations other than the above described equation can be used.

The establishment of the equation comprises determining the values of the parameters in the equation. The establishment of an equation for Tₘ calculation, i.e., determination of the values of the parameters in the equation for Tₘ calculation, is accomplished using each of the plurality of reference data sets including the information (i)-(ii).

The values of the parameters determined in this step will depend on the equation for Tₘ calculation used in step (b).

In one embodiment, the values of the parameters determined in step (b) comprises the value of the enthalpy change (ΔH) and the value of the entropy change (ΔS) for each of ten (10) nearest-neighbor (NN) sequences.

In another embodiment, the values of the parameters determined in step (b) comprises the value of the enthalpy change (ΔH) and the value of the entropy change (ΔS) for each of ten (10) nearest-neighbor (NN) sequences, e.g., “AA”, “AT”, “TA”, “GA”, “GT”, “GG”, “GC”, “CA”, “CT”, “CG”, and one or more additional parameters. The one or more additional parameters may comprise a parameter for correction of entropy change and/or a parameter for correction of Tₘ contribution by length of the oligonucleotide.

For example, where Equation I (in particular, Equation I-1) is used in the step (b) to calculate Tₘ, the values of the parameters determined in this step comprise the value of the enthalpy change (ΔH) and the value of the entropy change (ΔS) for each of ten (10) nearest-neighbor (NN) sequences, e.g., “AA”, “AT”, “TA”, “GA”, “GT”, “GG”, “GC”, “CA”, “CT”, “CG”. Since Equation I requires two NN parameters ΔH and ΔS for the Tₘ calculation, the values of the two parameters in Equation I should be determined in this step (b). It is reported that the number of all possible NN sequences is a total of 16 (4 bases×4 bases), but 6 NN sequences of them have the same parameter values. Thus, the values of the parameters to be determined in this step (b) are for the 10 NN sequences.

Where Equation III (in particular, Equation III-1) is used in the step (b) to calculate Tₘ, the values of the parameters determined in this step comprise the value of the enthalpy change (ΔH) and the value of the entropy change (ΔS) for each of ten (10) nearest-neighbor (NN) sequences, e.g., “AA”, “AT”, “TA”, “GA”, “GT”, “GG”, “GC”, “CA”, “CT”, “CG”, and the first additional parameter (a parameter for correction of entropy change). Since Equation III requires one additional parameter besides the two NN parameters ΔH and ΔS for the Tₘ calculation, a total of three parameters in Equation III should be determined in this step (b).

Where Equation IV (in particular, Equation IV-1) is used in the step (b) to calculate Tₘ, the values of the parameters determined in this step comprise the value of the enthalpy change (ΔH) and the value of the entropy change (ΔS) for each of ten (10) nearest-neighbor (NN) sequences, e.g., “AA”, “AT”, “TA”, “GA”, “GT”, “GG”, “GC”, “CA”, “CT”, “CG”, and the second additional parameter (a parameter for correction of Tₘ contribution by length of the oligonucleotide). Since Equation IV requires one additional parameter besides the two NN parameters ΔH and ΔS for the Tₘ calculation, a total of three parameters in Equation IV should be determined in this step (b).

Where Equation V (in particular, Equation V-1) is used in the step (b) to calculate Tₘ, the values of the parameters determined in this step comprise the value of the enthalpy change (ΔH) and the value of the entropy change (ΔS) for each of ten (10) nearest-neighbor (NN) sequences, e.g., “AA”, “AT”, “TA”, “GA”, “GT”, “GG”, “GC”, “CA”, “CT”, “CG”, and the first additional parameter (a parameter for correction of entropy change) and the second additional parameter (a parameter for correction of Tₘ contribution by length of the oligonucleotide). Since Equation V requires two additional parameters besides the two NN parameters ΔH and ΔS for the Tₘ calculation, a total of four parameters in Equation V should be determined in this step (b).

The determination of the values of the parameters in step (b) may be performed by linear regression or nonlinear regression. The determination of the values of the parameters means solving for 10 unknown.

In one embodiment, the determination of the values of the parameters in step (b) is performed by least square method. In an embodiment, the values of the parameters included in the equation are each determined for each NN sequence, by finding a value to minimize the sum of squared errors by least squares method.

The results of the determination of the values of the parameters in step (b) comprise the data of the thermodynamic parameters (in the case of Equations III, IV and V, further comprising one or more correction parameters), i.e., a table of thermodynamic parameters.

To date, a variety of NN tables have been published (Breslauer et al. (1986) Proc Natl Acad Sci USA 83: 3746-3750; Sugimoto et al. (1996), Nuc Acids Res 24: 4501-4505; Allawi and Santa Lucia Biochemistry 36: 10581-10594; SantaLucia & Hicks (2004), Annu. Rev. Biophys. Biomol. Struct 33: 415-440). However, the conventional NN tables propose the values of the NN parameters optimized for a fixed reaction environment, so that they are not suitable for use in calculating the Tₘ of oligonucleotides in various reaction environments.

In contrast, the present invention is well suited for calculating the Tₘ of an oligonucleotide in a variety of reaction environments using the values of the NN parameters optimized for each reaction environment.

As described above, Equation III, IV or V further comprises a first additional parameter for correcting the entropy change and/or a second additional parameter for correcting the oligonucleotide length, in addition to the NN parameters.

Some conventional equations have been developed, including a correction factor that reflects the salt concentration in the reaction environment or the length of an oligonucleotide. However, since the Tₘ of an oligonucleotide is also affected by many other factors as well as it is difficult to establish the equation for Tₘ calculation considering all of these correction factors, the approach using a correction factor is also not suitable for predicting an accurate Tₘ of an oligonucleotide that reflects the entire reaction environment. Further, the above method assumes that the values of the NN parameters for each NN sequence (also referred to as NN propagation) do not change for all reaction environments, except for the value of the correction factor. However, since such changes in the reaction environment also affect the values of the thermodynamic NN parameters for each NN sequence, the above approach has limitations in accurately predicting the Tₘ of the oligonucleotide in various reaction environments.

In contrast, according to the method of the present invention, the values of the additional parameters used in Equation III, IV or V as well as the values of the NN parameters vary depending on reaction environment. That is, the method of the present invention is distinct from the conventional methods, in terms of the variability in the values of the NN parameters and the value(s) of the additional parameter(s) depending on the reaction environment. The method of the present invention can accurately predict the change of Tₘ depending on varying reaction environment by using various parameters depending on the change in the reaction environment.

In one embodiment, the equation for Tₘ calculation used to determine the values of the parameters is the same for all reaction environments. For example, the equation for Tₘ calculation used in a first reference data set may be Equation V and the equation for Tₘ calculation used in a second reference data set may also be Equation V.

In another embodiment, the equation for Tₘ calculation used to determine the values of the parameters is different for some reaction environments. For example, the equation for Tₘ calculation used in a first reference data set may be Equation I, while the equation for Tₘ calculation used in a second reference data set may be Equation V.

In one embodiment, the values of the parameters determined are different for all reaction environments. For example, the value of the enthalpy change (ΔH) and the value of the entropy change (ΔS) for each of ten nearest-neighbor (NN) sequences and the value of the first additional parameter and the value of the second additional parameter determined for a first reference data set are different from those determined for a second reference data set. The difference in these parameter values for different reaction environments ultimately leads to the difference in the predicted Tₘ of an oligonucleotides of interest in different reaction environments.

### Step (c): Calculating the Tₘ of an Oligonucleotide of Interest in a Defined Reaction Environment

In this step, the Tₘ of an oligonucleotide of interest in one among the differently defined reaction environments is calculated using the equation established for the one among the differently defined reaction environments.

The established equations for Tₘ calculations for the differently defined reaction environments can be used to predict the Tₘ of an oligonucleotide of interest in various reaction environments.

Specifically, the equations having the parameter values determined for the differently defined reaction environments are provided in step (c).

In one embodiment, the values of the parameters comprise the value of the enthalpy change (ΔH) and the value of the entropy change (ΔS) for each of the ten nearest-neighbor (NN) sequences. In another embodiment, the values of the parameters comprise the value of the enthalpy change (ΔH) and the value of the entropy change (ΔS) for each of the nearest-neighbor (NN) sequence and the value of one or more additional parameters, wherein the one or more additional parameters comprise a parameter for correction of entropy change and/or a parameter for correction of length of the oligonucleotide.

In this step, the Tₘ of an oligonucleotide of interest in one among the differently defined reaction environments is calculated using the equation established for the one among the differently defined reaction environments.

The melting temperature of the oligonucleotide of interest calculated in this step is referred to herein as the “predicted Tₘ”, as it is determined by the equation established by the method of the present invention, rather than determined by any experimentation including melting analysis.

For example, the predicted Tₘ of an oligonucleotide of interest in a first reaction environment may be calculated by the equation for Tₘ calculation having the parameter values determined using a first reference data set (comprising information about the sequences of a plurality of reference oligonucleotides and information about the experimental Tₘs of the plurality of reference oligonucleotides in the first reaction environment); whereas the predicted Tₘ of an oligonucleotide of interest in a second reaction environment may be calculated by the equation for Tₘ calculation having the parameter values determined using a second reference data set (comprising information about the sequences of a plurality of reference oligonucleotides and information about the experimental Tₘs of the plurality of reference oligonucleotides in the second reaction environment).

The established equation for each of the differently defined reaction environments is used to calculate the melting temperature of an oligonucleotide of interest in the each of the differently defined reaction environments.

In one embodiment, where Equation I having the parameter values determined for a particular reference data set is established in step (b), the melting temperature of an oligonucleotide of interest is calculated as follows:

First, the N-1 NN sequences of an oligonucleotide of interest consisting of N nucleotides are provided. Thereafter, the values of the NN parameters for the N-1 NN sequences of an oligonucleotide of interest as determined in step (b) is applied to the above established Equation I to calculate the Tₘ of an oligonucleotide of interest.

In one embodiment, where Equation III, IV or V having the parameter values determined for a particular reference data set is established in step (b), the melting temperature of an oligonucleotide of interest is calculated as follows:

First, the N-1 NN sequences of an oligonucleotide of interest consisting of N nucleotides and the length N of the oligonucleotide (for Equations IV and V only) are provided. Thereafter, the values of the NN parameters for the N-1 NN sequences of an oligonucleotide of interest, and the value of the first additional parameter and/or the value of the second additional parameter, and the length N of the oligonucleotide (for Equations IV and V only) as determined in step (b) are applied to each established equation to calculate the Tₘ of an oligonucleotide of interest.

According to the method of the present invention, it is possible to accurately calculate the melting temperature of an oligonucleotide when used in various reaction environments. That is, the method of the present invention can accurately calculate not only the melting temperature of an oligonucleotide when present in a first reaction environment but also the melting temperature of the oligonucleotide when present in a second reaction environment.

According to the method of the present invention, the parameter values determined in a first reaction environment may be different from those in a second reaction environment, and thus the predicted Tₘ of the oligonucleotide in the first reaction environment may be different from that in the second reaction environment.

According to the method of the present invention, it is possible to establish various equations having the parameter values optimized for various reaction environments and to predict the Tₘ of an oligonucleotide of interest in a particular reaction environment by using a corresponding established equation. If there is no equation available for a particular reaction environment, the Tₘ of an oligonucleotide of interest can be predicted using an equation having the parameter values as determined in a similar reaction environment, or with slight modification of the predicted Tₘ.

### II. Method for Predicting the Melting Temperature (Tₘ) of an Oligonucleotide in a Defined Reaction Environment

In a second aspect of this invention, there is a method for predicting the melting temperature (Tₘ) of an oligonucleotide of interest in a defined environment, comprising the steps of:
(a) providing a reference data set, wherein the reference data set comprises (i) information about the sequences of a plurality of reference oligonucleotides and (ii) information about the experimental Tₘs of the plurality of reference oligonucleotides in a defined reaction environment;
(b) establishing an equation for Tₘ calculation for the defined reaction environment using the reference data set including the information (i)-(ii); wherein the establishment of the equation comprises determining the values of the parameters in the equation, wherein the parameters comprise nearest-neighbor (NN) thermodynamic parameters; and
(c) calculating the Tₘ of an oligonucleotide of interest in the defined reaction environment using the established equation for Tₘ calculation.

The second aspect of the present invention will now be described in detail.

### Step (a): Providing a Reference Data Set 110

First, a reference data set is provided 110. The reference data set comprises (i) information about the sequences of a plurality of reference oligonucleotides and (ii) information about the experimental Tₘs of the plurality of reference oligonucleotides in a defined reaction environment.

Details of the reference data set can be found in the section of the first aspect of the present invention.

According to this aspect, the method of the present invention uses a single reference data set, in order to predict the melting temperature of an oligonucleotide of interest in a defined reaction environment; wherein the reference data set corresponds to the defined reaction environment.

The reference data set is used herein to establish an NN model optimized for a defined reaction environment and to calculate the Tₘ of an oligonucleotide of interest in the defined reaction environment.

The reference data set comprises (i) information about the sequences of a plurality of reference oligonucleotides and (ii) information about the experimental Tₘs of the plurality of reference oligonucleotides in a defined reaction environment.

The components constituting the reference data set will be described in detail.

### (i) Information About the Sequences of a Plurality of Reference Oligonucleotides

The reference data set includes information about the sequences of a plurality of reference oligonucleotides. The information about the sequences of a plurality of reference oligonucleotides comprises information about nearest-neighbor (NN) sequences derived from a plurality of reference oligonucleotides.

The number of the sequences of the reference oligonucleotides included in the reference data set is not particularly limited, as long as all the values of the thermodynamic parameters for the nearest-neighbor (NN) sequences (also referred to as NN propagation, preferably a total of 10 NN sequences) can be determined using the sequences of the reference oligonucleotides. The number of the sequences of the reference oligonucleotides included in the reference data set is preferably as large as possible for accurate determination of the NN parameter values, but it may be adjusted to reduce the time required for determination of the NN parameter values, and the like. For example, the number of the sequences of the reference oligonucleotides in the reference data set may be at least 20, at least 30, or at least 50, up to 100, up to 300, up to 500, up to 1000, up to 3000, or 5000.

### (ii) Information About the Experimental Tₘs of the Plurality of Reference Oligonucleotides in a Defined Reaction Environment

The reference data set includes information about the experimental Tₘs of the plurality of reference oligonucleotides in a defined reaction environment.

As used herein, the term “experimental melting temperatures of a plurality of reference oligonucleotides in a defined reaction environment” refers to an experimental measure of melting temperature of each of the reference oligonucleotides in a defined reaction environment.

As used herein, the term “reaction environment” refers to one in which the oligonucleotide whose Tₘ is to be predicted (oligonucleotide of interest) is used/placed/applied. In other words, the term “reaction environment” refers to the medium, particularly chemical characteristics, surrounding an oligonucleotide. The reaction environment may be defined by the types and concentrations of the components used in a reaction except the oligonucleotide of interest. For example, when an oligonucleotide whose Tₘ is to be predicted is used in a PCR reaction, examples of the components can be used in the reaction include, without limitation, a buffers (for pH adjustment) such as Tris, Tricine, bis-Tricine, HEPES, EPPS, CHES and CAPSO; monovalent ions such as KCl, (NH₄)₂SO₄, NaCl, LiCl, NH₄Cl, NaN₃, CoCl₂, KOAc, KSO4, (Mn(OAc)₂ and NaOAc; divalent ions such as MgCl₂, MgSO₄, MgSO₄, and ZnCl₂; stabilizers such as BSA and gelatin; proteins such as T4 gene 32 protein and Et SSB; non-ionic detergents such as NP-40, Tween20, Triton X-100, Triton X-114, CHAPSO, IGEPAL, CHAPS, MEGA-8, Brij 35 and BRIJ 58; reducing agents such as DTT, b-mercaptoethanol and TCEP; compatible solutes such as glycerol, trehalose, sucrose, xylitol, melezitose, xylene cyanol, mannose, sorbitol, mannitol, betaine, ectoine and proline; sulfoxides such as DMSO; amides such as formamide; tetraalkyl-ammonium salts such as TMAC and TMAA, Polyhydroxyl alcohols such as propylene glycol and polyvinyl alcohol (PVA); chelating agent such as EDTA and EGTA; enzymes such as polymerases, dNTPs, and the like.

In one embodiment, the reaction environment may exclude a reaction environment comprising 1 M NaCl.

As used herein, the term “experimental melting temperature (Tₘ)” of a reference oligonucleotide refers to a Tₘ of a reference oligonucleotide which is determined experimentally in a defined reaction environment. The experimental Tₘ may be determined by melting curve analysis known in the art. As an example, the experimental Tₘ of a reference oligonucleotide may be determined by measuring the change in absorbance from the duplex between the oligonucleotide and its complement as a function of temperature. As another example, the experimental Tₘ of a reference oligonucleotide may be determined by measuring the change in fluorescence signal from the duplex between the oligonucleotide and its complement (e.g., by using an intercalating dye) as a function of temperature.

It is well known in the art that the Tₘ of an oligonucleotide vary depending on the reaction environment in which the oligonucleotide is used/placed/applied. For the purpose of accurately predicting the Tₘ of an oligonucleotide of interest in a desired reaction environment, the method of the present invention employs the experimental Tₘs of reference oligonucleotides when placed in the same reaction environment as the oligonucleotide of interest.

Thus, reference oligonucleotides for providing their sequences and Tₘs included in a reference data set may be selected from oligonucleotides having experimental Tₘs measured in the same reaction environment as the reaction environment in which an oligonucleotide of interest to be placed, or may be prepared by experimentally measuring the Tₘs of the reference oligonucleotides in the same reaction environment as the reaction environment in which an oligonucleotide of interest to be placed.

In one embodiment, the reference data set comprises a plurality of reference oligonucleotides such that each NN sequence occurs with the frequency of at least two.

As used herein, the term “nearest-neighbor (NN) sequence” refers to a sequence consisting of two adjacent nucleotides (dinucleotide) in an oligonucleotide. The term may be used interchangeably with “nearest-neighbors”, “nearest-neighbor base-pairs”, or “nearest-neighbor pairs”.

For example, in the case of an oligonucleotide sequence consisting of 13bases of 5′-ATTGCTTGCTTCG-3′ (SEQ ID NO: 1), the total number of nearest-neighbor (NN) sequences is seven (7), i.e., “AT”, “TT”, “TG”, “GC”, “CT”, “TC” and “CG”. In this case, it is noted that “TT” occurs with the frequency of 3, “TG” with the frequency of 2, “GC” with the frequency of 2, “CT” with the frequency of 2.

The nearest-neighbor (NN) sequences possible are sixteen (16) types (4 bases×4 bases=16), e.g., “AA”, “AT”, “AG”, “AC”, “TA”, “TT”, “TG”, “TC”, “GA”, “GT”, “GG”, “GC”, “CA”, “CT”, “CG” and “CC”. However, it is known that the dinucleotides “AA”, “CA”, “GT”, “CT”, “GA” and “GG” have the same NN parameter values as “TT”, “TG”, “AC”, “AG”, “TC” and “CC”, respectively. Thus, the total number of nearest-neighbor (NN) sequences having parameter values to be determined is ten (10).

For the reference data set, the frequency for each of the NN sequences refers to the number of NN sequences occurred in reference oligonucleotides of the reference data set. That is, the frequency for each of the NN sequences in the reference data set can be obtained by calculating the sum of the number of each NN sequence from entire reference oligonucleotides. For example, if the reference data set has two reference oligonucleotides such as 5′-ATGT-3′ and 5′-GTTA-3′, the reference data set is said to have the frequency of 1 for AT, 1 for GT, 2 for GT, 1 for TT, and 1 for TA.

In one embodiment, the reference data set comprises reference oligonucleotides such that such that each NN sequence occurs with the frequency of at least 2, at least 3, at least 4, at least 5, at least 7, at least 10, at least 15, at least 20, at least 30, or at least 40.

In one embodiment, the reference data set comprises at least one reference oligonucleotide having a Tₘ falling within each temperature interval within a temperature range. The lower limit of the temperature range may be 20, 25, 30, 35, 40, 45, 50, 55, 60, 65, 70° C., or more. The upper limit of the temperature range may be 40, 45, 50, 55, 60, 65, 70, 75, 80, 85, 90, 95, 100° C., or more. The each temperature interval within a temperature range may have an interval of 3, 5, 7, 10, 15, 20, 25, 30, 35 or 40° C. As an example, when each temperature interval has an interval of 5° C., 10° C., 15° C. or therebetween, the reference data set may comprise at least one reference oligonucleotide having a Tₘ falling within the each temperature interval. Specifically, when the temperature range consists of temperature intervals of 30-40° C., 40-50° C., 50-60° C., 60-70° C., and 70-80° C., the reference data set may comprise at least one reference oligonucleotide having an experimental Tₘ of 30-40° C., at least one reference oligonucleotide having an experimental Tₘ of 40-50° C., at least one reference oligonucleotide having an experimental Tₘ of 50-60° C., at least one reference oligonucleotide having an experimental Tₘ of 60-70° C., and at least one reference oligonucleotide having an experimental Tₘ of 70-80° C.

### Step (b): Establishment of a Defined Equation for Tₘ Calculation

Afterwards, an equation for Tₘ calculation for the defined reaction environment is established using the reference data set including the information (i)-(ii); wherein the establishment of the equation comprises determining the values of the parameters in the equation, wherein the parameters comprise nearest-neighbor (NN) thermodynamic parameters; and

[symbol]

In this step, the reference data set is used to establish an equation for Tₘ calculation for the reference data set, i.e., for the defined reaction environments, by determining the values of the parameters included in the equation.

The defined equation for Tₘ calculation refers to any equation based on an NN model using thermodynamic parameters. The equation may be one known in the art or a modification thereof. The predicted Tₘ of the oligonucleotide may be varied according to the equation used.

The equation for Tₘ calculation used in the present invention is one comprising nearest-neighbor (NN) thermodynamic parameters, or a modification thereof.

In an embodiment, the equation for Tₘ calculation comprises the parameters of the enthalpy change (ΔH) and the entropy change (ΔS) for each of nearest-neighbor (NN) sequences, and one or more additional parameters.

In an embodiment, the one or more additional parameters comprise a parameter for correction (or supplement) of entropy change and/or a parameter for correction (or supplement) of length of the oligonucleotide.

In an embodiment, the equation for Tₘ calculation is represented by the following Equation V.

$$Tm = \frac{Δ H ° \times m}{Δ S ° + α} + β ln ( length ) - n \qquad Equation V$$

wherein Tₘ is the melting temperature of the oligonucleotide; ΔH° is the sum of the enthalpy changes; ΔS° is the sum of the entropy changes; α is a first additional parameter for correction of entropy change; β is a second additional parameter for correction of length of the oligonucleotide; length is the length of the oligonucleotide; and m and n are constants.

In this equation, the first additional parameter α for correction of entropy change is one determined additionally in step (b) besides the thermodynamic parameters (ΔH° and ΔS°). The first additional parameter may be referred to as “first correction parameter” or “parameter associated with entropy change”. The value of the first additional parameter depends on the reaction environment.

In this equation, the second additional parameter β is one determined additionally in step (b) besides the thermodynamic parameters (ΔH° and ΔS°). The second additional parameter may be referred to as “second correction parameter”; “parameter associated with the length of the oligonucleotide sequence”, or “parameter for the relationship between the length and Tₘ of the oligonucleotide sequence”. The second additional parameter β is one for reflecting the influence of the reaction environment on the length of the oligonucleotide. The value of the second additional parameter depends on the reaction environment.

In a particular embodiment, m is 1000 and n is 273.15.

Details of the reference oligonucleotide sequences, their experimental Tₘs, and the defined equation for Tₘ calculation can be found in the section of the first aspect of the present invention.

### Step (c): Calculating the Tₘ of an Oligonucleotide of Interest in the Defined Reaction Environment

In this step, the Tₘ of an oligonucleotide of interest in the defined reaction environment is calculated using the established equation for Tₘ calculation.

The established equation for Tₘ calculation having the parameter values determined for the defined reaction environment is provided in step (c). The established equation for Tₘ calculation having the parameter values determined for the defined reaction environment can be used to predict the Tₘ of an oligonucleotide of interest in the defined reaction environment.

In one embodiment, the values of the parameters comprise the value of the enthalpy change (ΔH) and the value of the entropy change (ΔS) for each of the ten nearest-neighbor (NN) sequences. In another embodiment, the values of the parameters comprise the value of the enthalpy change (ΔH) and the value of the entropy change (ΔS) for each of the nearest-neighbor (NN) sequence and the value of one or more additional parameters, wherein the one or more additional parameters comprise a parameter for correction of entropy change and/or a parameter for correction of length of the oligonucleotide.

The calculation of the melting temperature can be found in the section of the first aspect of the present invention.

According to the second aspect of the present invention, it is possible to accurately calculate the melting temperature of an oligonucleotide when placed in a specific reaction environment.

### III. Storage Medium and Device for Predicting the Melting Temperature of an Oligonucleotide

In another aspect of this invention, there is provided a computer readable storage medium containing instructions to configure a processor to a method for predicting the melting temperature (Tₘ) of an oligonucleotide, comprising:
(a) receiving information about the sequence of an oligonucleotide of interest; and
(b) applying the information to one of established equations for Tₘ calculation, thereby calculating the Tₘ of an oligonucleotide of interest in one among differently defined reaction environments;
wherein the established equations for Tₘ calculation are provided by the method, comprising:
   providing a plurality of reference data sets, wherein the reference data set comprises (i) information about the sequences of a plurality of reference oligonucleotides and (ii) information about the experimental Tₘs of the plurality of reference oligonucleotides in a defined reaction environment, wherein the plurality of reference data sets are generated in differently defined reaction environments; and
   establishing an equation for Tₘ calculation for each of the differently defined reaction environments using the information (i)-(ii) included in each of the plurality of reference data sets; wherein the establishment of the equation comprises determining the values of the parameters in the equation, wherein the parameters comprise nearest-neighbor (NN) thermodynamic parameters.

In a further aspect of this invention, there is provided a device for predicting the melting temperature of an oligonucleotide, comprising (a) a computer processor and (b) the computer readable storage medium described above coupled to the computer processor.

Since the storage medium and the device of the prevent invention described hereinbelow are intended to perform the present methods in a computer, the common descriptions between them are omitted in order to avoid undue redundancy leading to the complexity of this specification.

The program instructions are operative, when performed by the processor, to cause the processor to perform the present method described above. The program instructions for predicting the melting temperature of an oligonucleotide of interest may comprise (i) an instruction to receive sequence information about an oligonucleotide of interest; and (b) an instruction to apply the information to an established equation for Tₘ calculation, thereby calculating the Tₘ of an oligonucleotide of interest.

In the program instructions, the established equation for Tₘ calculation is provided by the method of the first or second aspect of the present invention.

Further, the program instructions that perform the prediction of the Tₘ of the oligonucleotide may further comprise instructions to display the predicted melting temperature of an oligonucleotide of interest to the display device.

The present method described above is implemented in a processor, such as a processor in a stand-alone computer, a network attached computer or a data acquisition device such as a real-time PCR machine.

The types of the computer readable storage medium include various storage medium such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium such as a portable HDD, USB, floppy disk, CD and DVD. A computer code for implementing the present invention may be implemented in a variety of coding languages such as C, C++, Java, Visual Basic, VBScript, JavaScript, Perl and XML. In addition, a variety of languages and protocols may be used in external and internal storage and transmission of data and commands according to the present invention.

The processor may be prepared in such a manner that a single processor can do several performances. Alternatively, the processor unit may be prepared in such a manner that several processors do the several performances, respectively.

The features and advantages of this invention will be summarized as follows:

Conventional Tₘ prediction methods predict the Tₘ of an oligonucleotide in various reaction environments by using an equation having the NN parameter values optimized for a fixed reaction environment. However, the difference between the environment in which the NN parameter values are derived and the environment in which the oligonucleotide having the Tₘ to be predicted is used may lead to an inaccurate prediction of Tₘ of an oligonucleotide. In contrast, the method of the present invention can accurately predict the Tₘ of an oligonucleotide in various reaction environments by the use of the parameter values optimized for the corresponding reaction environments, which is useful in the field of molecular biology involving hybridization, particularly in PCR.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Prediction of Melting Temperature of Oligonucleotide Using Nearest-Neighbor (NN) Parameters Optimized for Specific Reaction Environment

A reference data set, comprising the sequences of a plurality of reference oligonucleotides and their experimental Tₘs measured in a specific reaction environment, was used to determine the values of the nearest-neighbor (NN) thermodynamic parameters included in a Tₘ prediction model. Then, the Tₘ prediction model having the determined NN parameter values was used to calculate the predicted Tₘ of an oligonucleotide of interest in the reaction environment. The predicted Tₘ means a Tₘ of an oligonucleotide expected to have in a specific reaction environment (particularly, the composition of a buffer containing an enzyme), which is calculated by a Tₘ prediction model. The predicted Tₘ may depend on the reaction environment in which an oligonucleotide is used.

In this Example, a known equation for Tₘ calculation based on the NN model was used as a Tₘ prediction model. The values of the parameters included in the equation were each determined for each NN sequence, by finding a value to minimize the sum of squared errors by least squares method. Then, the equation for Tₘ calculation having the parameter values thus determined was used to calculate the predicted Tₘ of an oligonucleotide of interest. Next, an error between the experimental Tₘ and the prediction Tₘ of an oligonucleotide of interest was calculated to evaluate the Tₘ prediction performance of the above equation. The values of the parameters as determined above were compared with values of the parameters previously known in the art.

### <1-1> Preparation of Oligonucleotides and Measurement of Tₘ

Ninety-five (95) oligonucleotides of varying sequences and lengths were prepared and their experimental Tₘs were determined by melting curve analysis.

Specifically, a hybridizing oligonucleotide complementary to each oligonucleotide was prepared, and the formation of a duplex between each oligonucleotide and its complementary hybridizing oligonucleotide was detected using an intercalating dye. The experimental Tₘ of each oligonucleotide was determined by melting the resulting duplex.

Briefly, the reaction was conducted in the final volume of 20 μl 0.05 uM of oligonucleotide for Tₘ measurement, 0.05 uM of hybridizing oligonucleotide, 1X EvaGreen (Biotium, USA) as an intercalating dye, and 5 μl of 4X Master Mix (final, 200 uM dNTPs, 2 mM MgCl₂, 2 U of Taq DNA polymerase) (Enzynomics, Korea); the tube containing the reaction mixture was placed in the real-time thermocycler (CFX96, Bio-Rad); the reaction mixture was subjected to melting curve analysis consisting of 5 minutes at 50° C., 15 minutes at 95° C., 10 minutes at 50° C., and slow heating from 50° C. to 85° C. by 0.5° C. The fluorescence was measured continuously during the temperature rise and the experimental Tₘs of the ninety-five (95) oligonucleotides were determined.

### <1-2> Providing Reference Data Set

Thirty-seven (37) oligonucleotides (about 39%) out of the 95 oligonucleotides having the experimental Tₘs were selected as reference oligonucleotides for calculation of NN parameters optimized for the reaction environment of this example. The reference oligonucleotides were selected such that each NN sequence occurs with the frequency of at least 20. The sequences and the melting temperatures of the reference oligonucleotides were pooled to prepare a reference data set.

For the reference data set, the length and experimental Tₘ of each reference oligonucleotide and the frequency for each NN sequence are shown in Table 1 below.

TABLE 1

| | | | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|---|---|
| | Ex. Tₘ | length | TT | TA | TC | TG | AT | AA | AC | AG | CT | CA | CC | CG | GT | GA | GC | GG |
| oligo1 | 53.8 | 17 | 2 | 2 | 1 | 1 | 0 | 0 | 2 | 0 | 4 | 0 | 1 | 0 | 1 | 0 | 1 | 1 |
| oligo2 | 54.5 | 23 | 1 | 3 | 0 | 1 | 3 | 7 | 1 | 1 | 0 | 1 | 0 | 0 | 1 | 1 | 0 | 2 |
| oligo3 | 55.3 | 20 | 2 | 2 | 0 | 1 | 2 | 2 | 2 | 0 | 1 | 1 | 1 | 1 | 1 | 1 | 1 | 1 |
| oligo4 | 57.0 | 17 | 2 | 1 | 0 | 0 | 0 | 4 | 0 | 1 | 1 | 0 | 2 | 2 | 0 | 1 | 2 | 0 |
| oligo5 | 57.3 | 18 | 2 | 2 | 1 | 1 | 0 | 0 | 2 | 0 | 4 | 0 | 1 | 0 | 1 | 0 | 1 | 2 |
| oligo6 | 57.5 | 21 | 2 | 2 | 0 | 1 | 2 | 2 | 2 | 0 | 1 | 1 | 1 | 1 | 1 | 1 | 1 | 2 |
| oligo7 | 58.0 | 21 | 1 | 1 | 2 | 1 | 2 | 0 | 0 | 3 | 0 | 1 | 1 | 2 | 2 | 3 | 1 | 0 |
| oligo8 | 58.5 | 18 | 0 | 0 | 0 | 2 | 1 | 2 | 1 | 1 | 0 | 0 | 0 | 3 | 1 | 3 | 2 | 1 |
| oligo9 | 58.5 | 27 | 1 | 5 | 1 | 1 | 3 | 5 | 1 | 3 | 0 | 2 | 0 | 0 | 3 | 1 | 0 | 0 |
| oligo10 | 59.0 | 19 | 2 | 1 | 2 | 0 | 0 | 1 | 2 | 1 | 2 | 0 | 1 | 2 | 1 | 2 | 0 | 1 |
| oligo11 | 59.0 | 19 | 0 | 0 | 2 | 0 | 1 | 3 | 3 | 0 | 1 | 3 | 2 | 1 | 0 | 2 | 0 | 0 |
| oligo12 | 59.0 | 27 | 0 | 2 | 2 | 0 | 2 | 9 | 2 | 2 | 1 | 2 | 0 | 1 | 1 | 2 | 0 | 0 |
| oligo13 | 60.0 | 26 | 1 | 3 | 0 | 1 | 3 | 7 | 1 | 2 | 0 | 1 | 0 | 0 | 1 | 2 | 1 | 2 |
| oligo14 | 60.0 | 21 | 3 | 2 | 1 | 1 | 1 | 0 | 2 | 1 | 3 | 1 | 1 | 1 | 0 | 0 | 2 | 1 |
| oligo15 | 60.0 | 21 | 3 | 2 | 1 | 1 | 1 | 0 | 2 | 1 | 3 | 1 | 1 | 1 | 0 | 0 | 2 | 1 |
| oligo16 | 60.0 | 21 | 4 | 2 | 2 | 1 | 0 | 0 | 1 | 1 | 4 | 0 | 2 | 0 | 0 | 0 | 1 | 2 |
| oligo17 | 60.0 | 21 | 4 | 2 | 2 | 1 | 0 | 0 | 1 | 1 | 4 | 0 | 2 | 0 | 0 | 0 | 1 | 2 |
| oligo18 | 60.0 | 20 | 0 | 2 | 2 | 1 | 0 | 3 | 0 | 3 | 2 | 0 | 2 | 0 | 2 | 1 | 1 | 0 |
| oligo19 | 60.3 | 22 | 2 | 2 | 0 | 1 | 2 | 2 | 2 | 1 | 1 | 1 | 1 | 1 | 1 | 1 | 1 | 2 |
| oligo20 | 61.0 | 20 | 0 | 0 | 1 | 2 | 0 | 5 | 1 | 1 | 2 | 2 | 1 | 0 | 1 | 1 | 2 | 0 |
| oligo21 | 61.0 | 22 | 5 | 2 | 2 | 1 | 0 | 0 | 2 | 1 | 2 | 0 | 1 | 1 | 2 | 1 | 0 | 1 |
| oligo22 | 61.8 | 22 | 5 | 2 | 1 | 2 | 0 | 0 | 2 | 0 | 4 | 0 | 1 | 0 | 1 | 0 | 1 | 2 |
| oligo23 | 62.0 | 23 | 2 | 0 | 2 | 1 | 2 | 3 | 3 | 0 | 1 | 3 | 2 | 1 | 0 | 2 | 0 | 0 |
| oligo24 | 62.5 | 20 | 0 | 0 | 0 | 3 | 2 | 2 | 1 | 1 | 0 | 0 | 0 | 3 | 1 | 3 | 2 | 1 |
| oligo25 | 62.5 | 20 | 0 | 0 | 2 | 0 | 1 | 2 | 4 | 0 | 2 | 3 | 2 | 1 | 0 | 2 | 0 | 0 |
| oligo26 | 63.0 | 25 | 4 | 3 | 2 | 1 | 1 | 1 | 1 | 2 | 2 | 2 | 0 | 0 | 3 | 0 | 1 | 1 |
| oligo27 | 63.3 | 21 | 1 | 3 | 0 | 1 | 1 | 0 | 1 | 3 | 1 | 1 | 2 | 1 | 2 | 1 | 2 | 0 |
| oligo28 | 63.5 | 21 | 0 | 0 | 2 | 1 | 1 | 2 | 4 | 0 | 1 | 3 | 2 | 2 | 0 | 2 | 0 | 0 |
| oligo29 | 64.0 | 22 | 0 | 2 | 2 | 1 | 0 | 3 | 1 | 3 | 3 | 0 | 2 | 0 | 2 | 1 | 1 | 0 |
| oligo30 | 65.5 | 29 | 4 | 2 | 1 | 1 | 1 | 4 | 1 | 2 | 2 | 1 | 0 | 0 | 2 | 1 | 0 | 4 |
| oligo31 | 66.5 | 25 | 2 | 3 | 1 | 2 | 1 | 0 | 3 | 1 | 0 | 1 | 2 | 2 | 4 | 1 | 0 | 1 |
| oligo32 | 67.0 | 21 | 3 | 1 | 1 | 2 | 0 | 0 | 1 | 0 | 2 | 0 | 3 | 2 | 2 | 0 | 2 | 1 |
| oligo33 | 69.0 | 27 | 1 | 2 | 1 | 4 | 2 | 1 | 0 | 1 | 1 | 0 | 0 | 1 | 4 | 1 | 1 | 6 |
| oligo34 | 69.0 | 25 | 1 | 1 | 1 | 2 | 1 | 1 | 1 | 2 | 0 | 1 | 1 | 3 | 3 | 3 | 1 | 2 |
| oligo35 | 69.0 | 25 | 1 | 0 | 2 | 2 | 1 | 1 | 1 | 2 | 0 | 1 | 1 | 3 | 3 | 3 | 1 | 2 |
| oligo36 | 69.5 | 26 | 3 | 2 | 2 | 1 | 0 | 1 | 3 | 1 | 2 | 0 | 0 | 3 | 3 | 2 | 1 | 1 |
| oligo37 | 71.5 | 25 | 1 | 0 | 0 | 2 | 0 | 1 | 4 | 2 | 0 | 3 | 4 | 1 | 2 | 3 | 1 | 0 |

| | | | | | | | | | | | | | | | | |
|---|---|---|---|---|---|---|---|---|---|---|---|---|---|---|---|---|
| Total | 65 | 59 | 42 | 45 | 37 | 74 | 61 | 44 | 57 | 36 | 43 | 40 | 52 | 48 | 34 | 42 |

(* The slash indicates the sequence are given in antiparallel orientation; e.g., AC/TG means 5′-AC-3′ is Watson-Crikc base paired with 3′-TG-5′)

### <1-3> Establishment of an Equation for Tₘ Calculation

The NN sequences and experimental Tₘs for the reference data set were applied to an equation for Tₘ calculation, thereby determining the values of the parameters included in the equation.

One of NN models using thermodynamic parameters ΔH° and ΔS° was used as the equation for Tₘ calculation (see SantaLucia, J. Jr (2007) Physical principles and visual-OMP software for optimal PCR design. Methods Mol. Biol., 402, 3-34). The equation is represented by the following Equation I-1.

$$Tm = \frac{Δ H ° \times 1000}{Δ S °} - 273.15 \qquad Equation I - 1$$

wherein Tₘ is the melting temperature of the oligonucleotide; ΔH° is the sum of the enthalpy changes; and ΔS° is the sum of the entropy changes.

In the above equation, ΔH° can be calculated by the sum of each NN parameter ΔH°_{NN}, and ΔS° can be calculated by the sum of each NN parameter ΔS°_{NN}, as shown in the following Equation II.

$$Δ H ° = {\sum }_{NN} ( each Δ H {°}_{NN} ) , Δ S ° = {\sum }_{NN} ( each Δ S {°}_{NN} ) \qquad Equation II$$

Each of the values of the NN parameters was calculated by finding a value to minimize the sum of squared errors by least square method. Among sixteen (16) NN sequences, six (6) NN sequences were assumed to have the same parameter values as their complementary NN sequences (AA/TT=TT/AA; CA/GT=TG/AC, GT/CA=AC/TG, CT/GA=AG/TC, GA/CT=TC/AG, GG/CC=CC/GG).

The NN parameter values for each NN sequence which are determined using the thirty-seven (37) reference oligonucleotides are shown in Table 2 below.

TABLE 2

| | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|
| Pair | TT | TA | TC | TG | AT | AA | AC | AG | CT | CA | CC | CG | GT | GA | GC | GG |
| ΔS° | −27.3 | −24.6 | −25.6 | −24.0 | −28.6 | −27.3 | −24.8 | −23.6 | −23.6 | −24.0 | −23.9 | −23.9 | −24.8 | −25.6 | −25.2 | −23.9 |
| ΔH° | −8.9 | −8.7 | −8.3 | −9.2 | −8.1 | −8.9 | −8.2 | −7.7 | −7.7 | −9.2 | −8.2 | −9.0 | −8.2 | −8.3 | −7.5 | −8.2 |

(*rounded to the nearest tenth)

### <1-4> Tₘ Prediction of Oligonucleotides Using the Equation Having the NN Parameter Values Optimized for the Specific Reaction Environment

The Equation I-1 having the values of the NN parameters determined in Example <1-3> was used to calculate the predicted Tₘs of the remaining fifty-eight (58) oligonucleotides. Then, the predicted Tₘ of each oligonucleotide was compared with the experimental Tₘ in Example 1 to evaluate the Tₘ prediction performance of the Equation I-1.

The experimental Tₘ, the NN parameters, the predicted Tₘ, and the error between the predicted Tₘ and the experimental Tₘ of each of the 58 oligonucleotides (oligo 38 to 95) are shown in Table 3 below.

TABLE 3

| | Ex. | Total | Total | Predicted | \|Predicted Tₘ − |
|---|---|---|---|---|---|
| | Tₘ | ΔH° | ΔS° | Tₘ | experimental Tₘ\| |
| oligo38 | 52.5 | −195.2 | −592.3 | 56.4 | 3.9 |
| oligo39 | 53.3 | −152.6 | −458.3 | 59.7 | 6.4 |
| oligo40 | 54.0 | −162.6 | −488.8 | 59.5 | 5.5 |
| oligo41 | 55.3 | −196.1 | −597.4 | 55.0 | 0.3 |
| oligo42 | 55.8 | −179.3 | −541.2 | 58.2 | 2.4 |
| oligo43 | 56.0 | −204.3 | −616.3 | 58.4 | 2.4 |
| oligo44 | 56.5 | −230.7 | −701.3 | 55.8 | 0.7 |
| oligo45 | 56.9 | −170.8 | −512.7 | 60.0 | 3.1 |
| oligo46 | 57.0 | −159.2 | −476.9 | 60.6 | 3.6 |
| oligo47 | 57.0 | −175.5 | −529.0 | 58.6 | 1.6 |
| oligo48 | 57.5 | −205.2 | −620.4 | 57.7 | 0.2 |
| oligo49 | 58.0 | −140.0 | −420.7 | 59.7 | 1.7 |
| oligo50 | 58.0 | −148.5 | −442.1 | 62.6 | 4.6 |
| oligo51 | 58.0 | −150.8 | −450.9 | 61.4 | 3.4 |
| oligo52 | 58.0 | −187.6 | −561.1 | 61.3 | 3.3 |
| oligo53 | 58.0 | −177.3 | −531.2 | 60.7 | 2.7 |
| oligo54 | 58.0 | −148.5 | −442.1 | 62.6 | 4.6 |
| oligo55 | 58.0 | −188.5 | −565.2 | 60.4 | 2.4 |
| oligo56 | 58.3 | −149.4 | −443.1 | 64.0 | 5.7 |
| oligo57 | 58.5 | −177.4 | −529.5 | 61.9 | 3.4 |
| oligo58 | 58.5 | −185.6 | −554.3 | 61.8 | 3.3 |
| oligo59 | 59.0 | −239.9 | −725.3 | 57.6 | 1.4 |
| oligo60 | 60.0 | −150.2 | −447.6 | 62.4 | 2.4 |
| oligo61 | 60.0 | −161.5 | −479.7 | 63.4 | 3.4 |
| oligo62 | 60.0 | −144.1 | −423.0 | 67.6 | 7.6 |
| oligo63 | 60.0 | −248.1 | −749.1 | 58.0 | 2.0 |
| oligo64 | 60.5 | −153.0 | −450.3 | 66.7 | 6.2 |
| oligo65 | 60.5 | −196.6 | −585.0 | 62.9 | 2.4 |
| oligo66 | 60.5 | −230.2 | −696.8 | 57.2 | 3.3 |
| oligo67 | 60.8 | −204.6 | −613.1 | 60.5 | 0.3 |
| oligo68 | 61.4 | −204.6 | −614.4 | 59.8 | 1.6 |
| oligo69 | 61.5 | −171.1 | −505.6 | 65.3 | 3.8 |
| oligo70 | 61.5 | −265.0 | −798.6 | 58.6 | 2.9 |
| oligo71 | 61.8 | −179.8 | −536.6 | 61.9 | 0.1 |
| oligo72 | 62.0 | −161.3 | −475.8 | 65.9 | 3.9 |
| oligo73 | 62.0 | −179.2 | −534.2 | 62.4 | 0.4 |
| oligo74 | 62.5 | −182.7 | −549.8 | 59.2 | 3.3 |
| oligo75 | 62.5 | −204.9 | −614.6 | 60.2 | 2.3 |
| oligo76 | 63.0 | −169.3 | −499.6 | 65.7 | 2.7 |
| oligo77 | 63.0 | −170.7 | −499.9 | 68.4 | 5.4 |
| oligo78 | 63.0 | −238.3 | −720.7 | 57.6 | 5.4 |
| oligo79 | 63.5 | −194.7 | −579.0 | 63.1 | 0.4 |
| oligo80 | 63.5 | −179.6 | −527.1 | 67.5 | 4.0 |
| oligo81 | 63.8 | −213.6 | −639.2 | 61.0 | 2.8 |
| oligo82 | 64.0 | −171.2 | −503.1 | 67.1 | 3.1 |
| oligo83 | 64.0 | −168.0 | −498.4 | 63.9 | 0.1 |
| oligo84 | 64.0 | −178.2 | −526.8 | 65.1 | 1.1 |
| oligo85 | 64.0 | −188.5 | −554.4 | 66.8 | 2.8 |
| oligo86 | 65.0 | −194.0 | −578.7 | 62.2 | 2.8 |
| oligo87 | 65.0 | −169.4 | −502.0 | 64.2 | 0.8 |
| oligo88 | 65.5 | −187.1 | −554.1 | 64.5 | 1.0 |
| oligo89 | 65.5 | −205.3 | −607.6 | 64.7 | 0.8 |
| oligo90 | 65.5 | −179.4 | −527.1 | 67.2 | 1.7 |
| oligo91 | 66.5 | −228.4 | −678.2 | 63.7 | 2.8 |
| oligo92 | 69.0 | −221.7 | −650.9 | 67.4 | 1.6 |
| oligo93 | 69.0 | −194.2 | −574.3 | 65.0 | 4.0 |
| oligo94 | 69.5 | −202.6 | −599.7 | 64.6 | 4.9 |
| oligo95 | 70.0 | −194.1 | −573.6 | 65.2 | 4.8 |

| | |
|---|---|
| Mean Absolute Error | 2.9 |
| Sum of Absolute Errors | 165.5 |
| Sum of Squared Errors | 647.7 |

(*rounded to the nearest tenth)

As shown in Table 3, the Tₘs of the 58 oligonucleotides were predicted with the mean absolute error of 2.9, the sum of ab solute error of 165.5, and the sum of squared error of 647.7.

### <1-5> Tₘ Prediction of Oligonucleotides Using the Equation Having Known NN Parameter Values

The Tₘ prediction performance of the equation having the NN parameter values determined in Example <1-4> was compared with the equation having known NN parameter values (SantaLucia & Hicks (2004), Annu. Rev. Biophys. Biomol. Struct 33: 415-440).

The known NN parameter values are shown in Table 4 below.

TABLE 4

| | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|
| Pair | TT | TA | TC | TG | AT | AA | AC | AG | CT | CA | CC | CG | GT | GA | GC | GG |
| ΔS° | −21.3 | −20.4 | −21.0 | −22.4 | −21.3 | −21.3 | −22.7 | −22.2 | −22.2 | −22.4 | −19.9 | −24.4 | −22.7 | −21.0 | −27.2 | −19.9 |
| ΔH° | −7.6 | −7.2 | −7.8 | −8.4 | −7.2 | −7.6 | −8.5 | −8.2 | −8.2 | −8.4 | −8.0 | −9.8 | −8.5 | −7.8 | −10.6 | −8.0 |

The predicted Tₘs of the 58 oligonucleotides were calculated in the same manner to Example <1-4> except for using the equation having the known NN parameter values.

Afterwards, the predicted Tₘ of each oligonucleotide was compared with the experimental Tₘ to evaluate the Tₘ prediction performance of the known values of the NN parameters.

The experimental Tₘ, the NN parameters, the predicted Tₘ, and the error between the predicted Tₘ and the experimental Tₘ of each of the 58 oligonucleotides (oligo 38 to 95) are shown in Table 5 below.

TABLE 5

| | Ex. | Total | Total | Predicted | \|Predicted Tₘ − |
|---|---|---|---|---|---|
| | Tₘ | ΔH° | ΔS° | Tₘ | experimental Tₘ\| |
| oligo38 | 52.5 | −178.5 | −494.8 | 87.6 | 35.1 |
| oligo39 | 53.3 | −146.1 | −395.1 | 96.6 | 43.3 |
| oligo40 | 54.0 | −152.4 | −413.8 | 95.1 | 41.1 |
| oligo41 | 55.3 | −180.4 | −495.9 | 90.6 | 35.3 |
| oligo42 | 55.8 | −163.6 | −446.7 | 93.1 | 37.3 |
| oligo43 | 56.0 | −186.9 | −517.2 | 88.2 | 32.2 |
| oligo44 | 56.5 | −208.9 | −580.0 | 87.0 | 30.5 |
| oligo45 | 56.9 | −160.4 | −433.7 | 96.7 | 39.8 |
| oligo46 | 57.0 | −152.6 | −410.6 | 98.5 | 41.5 |
| oligo47 | 57.0 | −167.5 | −457.9 | 92.7 | 35.7 |
| oligo48 | 57.5 | −189.3 | −519.5 | 91.2 | 33.7 |
| oligo49 | 58.0 | −145.2 | −386.0 | 103.0 | 45.0 |
| oligo50 | 58.0 | −150.5 | −401.1 | 101.3 | 43.3 |
| oligo51 | 58.0 | −149.4 | −400.2 | 100.2 | 42.2 |
| oligo52 | 58.0 | −173.0 | −472.4 | 93.1 | 35.1 |
| oligo53 | 58.0 | −169.8 | −459.9 | 96.1 | 38.1 |
| oligo54 | 58.0 | −150.2 | −401.1 | 101.3 | 43.3 |
| oligo55 | 58.0 | −172.0 | −469.1 | 93.5 | 35.5 |
| oligo56 | 58.3 | −147.1 | −393.0 | 101.2 | 42.9 |
| oligo57 | 58.5 | −168.6 | −456.8 | 95.9 | 37.4 |
| oligo58 | 58.5 | −176.7 | −481.2 | 94.1 | 35.6 |
| oligo59 | 59.0 | −217.3 | −602.4 | 87.6 | 28.6 |
| oligo60 | 60.0 | −150.3 | −400.0 | 102.6 | 42.6 |
| oligo61 | 60.0 | −153.9 | −413.3 | 99.2 | 39.2 |
| oligo62 | 60.0 | −141.2 | −374.4 | 104.0 | 44.0 |
| oligo63 | 60.0 | −225.3 | −622.3 | 88.9 | 28.9 |
| oligo64 | 60.5 | −148.8 | −395.7 | 102.9 | 42.4 |
| oligo65 | 60.5 | −182.8 | −496.8 | 94.8 | 34.3 |
| oligo66 | 60.5 | −212.9 | −584.0 | 91.4 | 30.9 |
| oligo67 | 60.8 | −190.5 | −518.3 | 94.4 | 33.6 |
| oligo68 | 61.4 | −188.0 | −512.3 | 93.8 | 32.4 |
| oligo69 | 61.5 | −161.9 | −434.6 | 99.4 | 37.9 |
| oligo70 | 61.5 | −241.0 | −665.4 | 89.0 | 27.5 |
| oligo71 | 61.8 | −170.2 | −458.1 | 98.4 | 36.6 |
| oligo72 | 62.0 | −156.6 | −416.7 | 102.7 | 40.7 |
| oligo73 | 62.0 | −169.1 | −455.9 | 97.8 | 35.8 |
| oligo74 | 62.5 | −181.0 | −484.1 | 100.7 | 38.2 |
| oligo75 | 62.5 | −191.1 | −518.9 | 95.1 | 32.6 |
| oligo76 | 63.0 | −163.4 | −436.9 | 100.8 | 37.8 |
| oligo77 | 63.0 | −163.6 | −437.1 | 101.1 | 38.1 |
| oligo78 | 63.0 | −220.9 | −603.9 | 92.6 | 29.6 |
| oligo79 | 63.5 | −186.2 | −502.2 | 97.6 | 34.1 |
| oligo80 | 63.5 | −171.2 | −458.4 | 100.3 | 36.8 |
| oligo81 | 63.8 | −198.3 | −539.3 | 94.5 | 30.7 |
| oligo82 | 64.0 | −167.1 | −446.7 | 100.9 | 36.9 |
| oligo83 | 64.0 | −163.8 | −436.7 | 101.9 | 37.9 |
| oligo84 | 64.0 | −171.0 | −458.2 | 100.0 | 36.0 |
| oligo85 | 64.0 | −178.8 | −479.7 | 99.6 | 35.6 |
| oligo86 | 65.0 | −187.6 | −506.4 | 97.3 | 32.3 |
| oligo87 | 65.0 | −167.8 | −448.8 | 100.7 | 35.7 |
| oligo88 | 65.5 | −178.6 | −479.5 | 99.3 | 33.8 |
| oligo89 | 65.5 | −193.2 | −521.4 | 97.4 | 31.9 |
| oligo90 | 65.5 | −172.6 | −460.4 | 101.7 | 36.2 |
| oligo91 | 66.5 | −218.8 | −589.5 | 98.0 | 31.5 |
| oligo92 | 69.0 | −214.4 | −571.7 | 101.9 | 32.9 |
| oligo93 | 69.0 | −192.8 | −509.2 | 105.5 | 36.5 |
| oligo94 | 69.5 | −200.8 | −531.7 | 104.5 | 35.0 |
| oligo95 | 70.0 | −193.5 | −510.9 | 105.6 | 35.6 |

| | |
|---|---|
| Mean Absolute Error | 36.3 |
| Sum of Absolute Errors | 2105.4 |
| Sum of Squared Errors | 77454.9 |

(*rounded to the nearest tenth)

As shown in Table 5, the Tₘs of the 58 oligonucleotides were predicted using known NN parameter values, with the mean absolute error of 36.3, the sum of absolute error of 2105.4, and the sum of squared error of 77454.9.

The Tₘ prediction performance of the equation having the NN parameter values optimized for the specific reaction environment according to the present invention (Table 3) was compared with that of the equation having the known NN parameter values (Table 5). As a result, it was found that the use of the equation having the NN parameter values (Table 3) optimized for a specific reaction environment according to the present invention reduced the mean absolute error by −33.4, the sum of absolute error by −1939.2, and the sum of squared error by of −76807.2.

The results demonstrate that using the optimized values of the NN parameters for a specific reaction environment allows for prediction of a more accurate Tₘ compared to using the known NN parameter values. The known NN parameter values are optimized for a fixed reaction environment (1M NaCl, pH 7), thereby resulting in unsuitability for use in different reaction environments.

Furthermore, for a sophisticated comparison between the NN parameter values determined according to the present invention and the known NN parameter values, the slope of the trend line and the coefficient of determination R² were determined in a scatter plot indicating the correlation between the experimental Tₘ and the predicted Tₘ.

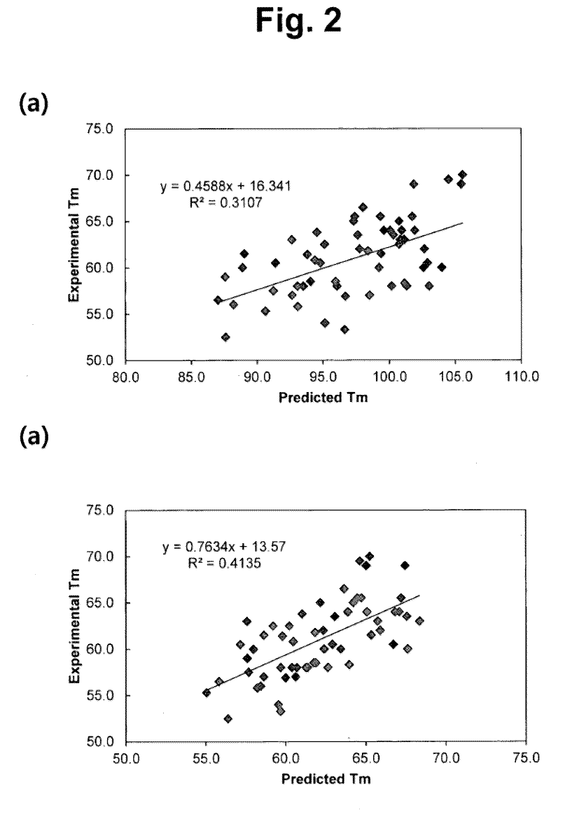

The results are shown in FIG. 2.

As shown in FIG. 2, the Tₘ prediction using the known NN parameter values (Table 5) showed the slope of the trend line of 0.4588 and the coefficient of determination R² of 0.3107, indicating a low correlation between the predicted Tₘ and the experimental Tₘ. In contrast, the Tₘ prediction using the NN parameter values according to the present invention showed the slope of the trend line of 0.7634 and the coefficient of determination R² of 0.4135, indicating a high correlation between the predicted Tₘ and the experimental Tₘ.

Considering that a higher correlation is associated with a higher similarity between the predicted Tₘ and the experimental Tₘ, the results demonstrate that using the NN parameter values optimized for the reaction environment makes it possible to more accurately predict the Tₘ of the oligonucleotide.

### Example 2: Prediction of Melting Temperature of Oligonucleotide Using a Modified Tₘ Prediction Model

In this Example, it was verified whether the accuracy of Tₘ prediction of the oligonucleotide can be improved by using other equations than Equation I-1.

In addition to the NN parameters, one or more additional parameters were introduced into Equation I-1 to generate modified equations for Tₘ calculation, and then the Tₘ prediction performances of these equations were determined.

Specifically, the following four (4) equations for Tₘ calculation were used: (i) an equation including only NN parameters (control group); (ii) an equation including a first additional parameter α in addition to the NN parameters; (iii) an equation including a second additional parameter β in addition to the NN parameters; and (iv) an equation including the first and second additional parameters α and β in addition to the NN parameters.

As in Example 1, a reference data set comprising thirty-seven (37) reference oligonucleotides was used to determine the parameter values that minimized the sum of squared errors for each of the four (4) equations. Then, each of the four equations having the parameter values was used to calculate the predicted Tₘs of fifty-eight (58) oligonucleotides.

### <2-1> Tₘ Prediction of Oligonucleotide Using an Equation Including Only NN Parameters

Equation I-1 was used as an equation for Tₘ calculation, including only the NN parameters. The Tₘs of fifty-eight (58) oligonucleotides were predicted using Equation I-1 having the NN parameter values as shown in Table 2. The predicted Tₘs of the 58 oligonucleotides are shown in Table 3.

### <2-2> Tₘ Prediction Using an Equation Including the NN Parameters and the Parameter α

A first additional parameter α was added to Equation I-1 to generate a modified equation for Tₘ calculation, Equation III-1.

$$Tm = \frac{Δ H ° \times 1000}{Δ S ° + α} - 273.15 \qquad Equation III ‐ 1$$

Thirty-seven (37) reference oligonucleotides of Example <1-2> were used to determine the values of the NN parameters and the value of the parameter a for Equation III-1 above.

The values of the NN parameters determined were shown in Table 6, and the value of the parameter α determined was −24.9.

TABLE 6

| | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|
| Pair | TT | TA | TC | TG | AT | AA | AC | AG | CT | CA | CC | CG | GT | GC | GA | GG |
| ΔS° | −23.5 | −25.1 | −23.8 | −23.4 | −25.2 | −23.5 | −23.9 | −25.4 | −25.4 | −23.4 | −21.3 | −26.1 | −23.9 | −23.8 | −24.5 | −21.3 |
| ΔH° | −8.0 | −8.1 | −8.3 | −8.2 | −8.0 | −8.0 | −8.8 | −8.9 | −8.9 | −8.2 | −7.9 | −9.4 | −8.8 | −8.3 | −9.2 | −7.9 |

(*rounded to the nearest tenth)

The Equation III-1 having the parameter values above was used to calculate the predicted Tₘs of fifty-eight (58) oligonucleotides (oligo 38-95).

The experimental Tₘ, the NN parameters, the predicted Tₘ, and the error between the predicted Tₘ and the experimental Tₘ of each of the 58 oligonucleotides (oligo 38 to 95) are shown in Table 7 below.

TABLE 7

| | Ex. | Total | Total | Predicted | \|Predicted Tₘ − |
|---|---|---|---|---|---|
| | Tₘ | ΔH° | ΔS° | Tₘ | experimental Tₘ\| |
| oligo38 | 52.5 | −190.6 | −559.3 | 53.1 | 0.6 |
| oligo39 | 53.3 | −150.5 | −431.7 | 56.4 | 3.1 |
| oligo40 | 54.0 | −159.0 | −458.1 | 56.0 | 2.0 |
| oligo41 | 55.3 | −188.7 | −549.8 | 55.2 | 0.1 |
| oligo42 | 55.8 | −171.9 | −496.7 | 56.5 | 0.7 |
| oligo43 | 56.0 | −198.8 | −582.7 | 54.1 | 1.9 |
| oligo44 | 56.5 | −222.6 | −653.3 | 55.1 | 1.4 |
| oligo45 | 56.9 | −166.9 | −479.4 | 57.8 | 0.9 |
| oligo46 | 57.0 | −160.2 | −456.1 | 59.9 | 2.9 |
| oligo47 | 57.0 | −176.3 | −511.1 | 55.8 | 1.2 |
| oligo48 | 57.5 | −199.5 | −579.1 | 57.2 | 0.3 |
| oligo49 | 58.0 | −145.9 | −412.0 | 60.9 | 2.9 |
| oligo50 | 58.0 | −154.0 | −438.3 | 59.3 | 1.3 |
| oligo51 | 58.0 | −154.0 | −437.8 | 59.7 | 1.7 |
| oligo52 | 58.0 | −181.8 | −524.7 | 57.7 | 0.3 |
| oligo53 | 58.0 | −178.4 | −511.9 | 59.1 | 1.1 |
| oligo54 | 58.0 | −154.0 | −438.3 | 59.3 | 1.3 |
| oligo55 | 58.0 | −180.1 | −520.1 | 57.4 | 0.6 |
| oligo56 | 58.3 | −151.9 | −429.7 | 61.1 | 2.8 |
| oligo57 | 58.5 | −176.8 | −506.8 | 59.3 | 0.8 |
| oligo58 | 58.5 | −184.4 | −531.2 | 58.5 | 0.0 |
| oligo59 | 59.0 | −230.9 | −676.7 | 55.9 | 3.1 |
| oligo60 | 60.0 | −154.6 | −436.5 | 61.9 | 1.9 |
| oligo61 | 60.0 | −158.6 | −451.9 | 59.6 | 0.4 |
| oligo62 | 60.0 | −144.3 | −406.0 | 61.7 | 1.7 |
| oligo63 | 60.0 | −238.8 | −698.1 | 57.1 | 2.9 |
| oligo64 | 60.5 | −152.3 | −429.5 | 62.1 | 1.6 |
| oligo65 | 60.5 | −191.3 | −550.8 | 59.1 | 1.4 |
| oligo66 | 60.5 | −224.7 | −651.8 | 59.0 | 1.5 |
| oligo67 | 60.8 | −199.4 | −574.7 | 59.5 | 1.3 |
| oligo68 | 61.4 | −197.4 | −569.3 | 59.0 | 2.4 |
| oligo69 | 61.5 | −166.6 | −473.7 | 61.0 | 0.5 |
| oligo70 | 61.5 | −255.6 | −747.1 | 58.0 | 3.5 |
| oligo71 | 61.8 | −176.3 | −505.5 | 59.3 | 2.5 |
| oligo72 | 62.0 | −160.6 | −453.3 | 62.7 | 0.7 |
| oligo73 | 62.0 | −174.6 | −498.9 | 60.3 | 1.7 |
| oligo74 | 62.5 | −187.2 | −533.4 | 62.2 | 0.3 |
| oligo75 | 62.5 | −198.8 | −572.7 | 59.5 | 3.0 |
| oligo76 | 63.0 | −168.3 | −476.0 | 62.8 | 0.2 |
| oligo77 | 63.0 | −167.6 | −474.0 | 62.7 | 0.3 |
| oligo78 | 63.0 | −232.6 | −673.1 | 60.2 | 2.8 |
| oligo79 | 63.5 | −194.5 | −556.6 | 61.3 | 2.2 |
| oligo80 | 63.5 | −175.6 | −497.5 | 62.9 | 0.6 |
| oligo81 | 63.8 | −206.8 | −597.8 | 59.0 | 4.8 |
| oligo82 | 64.0 | −169.9 | −479.5 | 63.7 | 0.3 |
| oligo83 | 64.0 | −170.1 | −480.8 | 63.2 | 0.8 |
| oligo84 | 64.0 | −176.3 | −499.5 | 63.0 | 1.0 |
| oligo85 | 64.0 | −183.6 | −521.0 | 63.1 | 0.9 |
| oligo86 | 65.0 | −194.2 | −554.7 | 61.9 | 3.1 |
| oligo87 | 65.0 | −169.6 | −479.1 | 63.4 | 1.6 |
| oligo88 | 65.5 | −184.3 | −523.0 | 63.2 | 2.3 |
| oligo89 | 65.5 | −199.7 | −571.3 | 61.8 | 3.7 |
| oligo90 | 65.5 | −176.8 | −500.2 | 63.6 | 1.9 |
| oligo91 | 66.5 | −227.7 | −647.3 | 65.6 | 0.9 |
| oligo92 | 69.0 | −220.6 | −623.0 | 67.3 | 1.7 |
| oligo93 | 69.0 | −195.4 | −548.9 | 67.5 | 1.5 |
| oligo94 | 69.5 | −204.1 | −574.1 | 67.6 | 1.9 |
| oligo95 | 70.0 | −195.9 | −549.0 | 68.3 | 1.7 |

| | |
|---|---|
| Mean Absolute Error | 1.6 |
| Sum of Absolute Errors | 92.3 |
| Sum of Squared Errors | 188.7 |

(*rounded to the nearest tenth)

As shown in Table 7, the Tₘs of the 58 oligonucleotides were predicted using Equation III-1, with the mean absolute error of 1.6, the sum of absolute error of 92.3, and the sum of squared error of 188.7.

### <2-3> Tₘ Prediction Using an Equation Including the NN Parameters and the Parameter β

A second additional parameter β was added to Equation I-1 to generate a modified equation for Tₘ calculation, Equation IV-1.

$$Tm = \frac{Δ H ° \times 1000}{Δ S °} + β ln ( length ) - 273.15 \qquad Equation IV ‐ 1$$

Thirty-seven (37) reference oligonucleotides of Example <1-2> were used to determine the values of the NN parameters and the value of the parameter β for Equation IV-1 above.

The values of the NN parameters determined were shown in Table 8, and the value of the parameter β determined was 5.00.

TABLE 8

| | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|
| Pair | TT | TA | TC | TG | AT | AA | AC | AG | CT | CA | CC | CG | GT | GA | GC | GG |
| ΔS° | −27.2 | −26.2 | −24.6 | −23.4 | −27.0 | −27.2 | −25.1 | −25.1 | −25.1 | −23.4 | −24.2 | −25.4 | −25.1 | −24.6 | −24.6 | −24.2 |
| ΔH° | −8.4 | −8.6 | −7.6 | −8.4 | −7.2 | −8.4 | −7.9 | −7.9 | −7.9 | −8.4 | −8.0 | −9.0 | −7.9 | −7.6 | −7.1 | −8.0 |

(*rounded to the nearest tenth)

The Equation IV-1 having the parameter values above was used to calculate the predicted Tₘs of fifty-eight (58) oligonucleotides (oligo 38-95).

The experimental Tₘ, the NN parameters, the predicted Tₘ, and the error between the predicted Tₘ and the experimental Tₘ of each of the 58 oligonucleotides (oligo 38 to 95) are shown in Table 9 below.

TABLE 9

| | Ex. | Total | Total | Predicted | \|Predicted Tₘ − |
|---|---|---|---|---|---|
| | Tₘ | ΔH° | ΔS° | Tₘ | experimental Tₘ\| |
| oligo1 | 52.5 | −186.6 | −595.7 | 52.5 | 3.5 |
| oligo2 | 53.3 | −146.2 | −460.7 | 53.3 | 5.5 |
| oligo3 | 54.0 | −155.3 | −490.2 | 54.0 | 4.7 |
| oligo4 | 55.3 | −186.5 | −596.9 | 55.3 | 0.1 |
| oligo5 | 55.8 | −172.1 | −545.4 | 55.8 | 2.1 |
| oligo6 | 56.0 | −195.1 | −619.1 | 56.0 | 2.0 |
| oligo7 | 56.5 | −220.4 | −704.5 | 56.5 | 0.2 |
| oligo8 | 56.9 | −163.3 | −514.5 | 56.9 | 2.6 |
| oligo9 | 57.0 | −154.3 | −484.7 | 57.0 | 3.2 |
| oligo10 | 57.0 | −169.4 | −536.4 | 57.0 | 1.2 |
| oligo11 | 57.5 | −195.3 | −620.2 | 57.5 | 0.3 |
| oligo12 | 58.0 | −135.8 | −426.5 | 58.0 | 1.6 |
| oligo13 | 58.0 | −144.4 | −450.7 | 58.0 | 3.9 |
| oligo14 | 58.0 | −145.7 | −456.5 | 58.0 | 2.8 |
| oligo15 | 58.0 | −180.3 | −566.5 | 58.0 | 2.7 |
| oligo16 | 58.0 | −170.7 | −536.6 | 58.0 | 2.4 |
| oligo17 | 58.0 | −144.4 | −450.7 | 58.0 | 3.9 |
| oligo18 | 58.0 | −180.6 | −568.8 | 58.0 | 2.0 |
| oligo19 | 58.3 | −145.2 | −451.3 | 58.3 | 4.9 |
| oligo20 | 58.5 | −170.5 | −534.2 | 58.5 | 3.0 |
| oligo21 | 58.5 | −178.9 | −561.0 | 58.5 | 2.9 |
| oligo22 | 59.0 | −228.8 | −727.9 | 59.0 | 0.9 |
| oligo23 | 60.0 | −145.1 | −452.7 | 60.0 | 2.0 |
| oligo24 | 60.0 | −152.2 | −474.6 | 60.0 | 2.6 |
| oligo25 | 60.0 | −137.4 | −422.8 | 60.0 | 6.3 |
| oligo26 | 60.0 | −236.8 | −752.1 | 60.0 | 1.3 |
| oligo27 | 60.5 | −145.9 | −450.0 | 60.5 | 5.2 |
| oligo28 | 60.5 | −189.2 | −591.8 | 60.5 | 2.0 |
| oligo29 | 60.5 | −219.3 | −697.2 | 60.5 | 2.5 |
| oligo30 | 60.8 | −195.7 | −616.1 | 60.8 | 0.3 |
| oligo31 | 61.4 | −196.1 | −618.6 | 61.4 | 1.4 |
| oligo32 | 61.5 | −161.9 | −502.0 | 61.5 | 3.0 |
| oligo33 | 61.5 | −253.4 | −803.4 | 61.5 | 2.0 |
| oligo34 | 61.8 | −172.3 | −539.8 | 61.8 | 0.4 |
| oligo35 | 62.0 | −153.5 | −474.6 | 62.0 | 3.2 |
| oligo36 | 62.0 | −169.1 | −529.0 | 62.0 | 0.0 |
| oligo37 | 62.5 | −175.1 | −551.8 | 62.5 | 2.6 |
| oligo38 | 62.5 | −193.4 | −609.4 | 62.5 | 2.1 |
| oligo39 | 63.0 | −160.8 | −497.8 | 63.0 | 2.2 |
| oligo40 | 63.0 | −161.4 | −496.1 | 63.0 | 4.4 |
| oligo41 | 63.0 | −227.2 | −721.4 | 63.0 | 4.3 |
| oligo42 | 63.5 | −187.1 | −584.2 | 63.5 | 0.5 |
| oligo43 | 63.5 | −169.8 | −523.3 | 63.5 | 3.3 |
| oligo44 | 63.8 | −202.1 | −635.6 | 63.8 | 2.7 |
| oligo45 | 64.0 | −164.4 | −507.0 | 64.0 | 2.4 |
| oligo46 | 64.0 | −162.3 | −504.6 | 64.0 | 0.3 |
| oligo47 | 64.0 | −169.3 | −525.0 | 64.0 | 0.7 |
| oligo48 | 64.0 | −178.2 | −550.5 | 64.0 | 2.3 |
| oligo49 | 65.0 | −186.0 | −582.2 | 65.0 | 2.7 |
| oligo50 | 65.0 | −162.0 | −503.2 | 65.0 | 0.9 |
| oligo51 | 65.5 | −177.7 | −552.2 | 65.5 | 1.2 |
| oligo52 | 65.5 | −194.1 | −603.7 | 65.5 | 1.0 |
| oligo53 | 65.5 | −170.3 | −525.2 | 65.5 | 1.1 |
| oligo54 | 66.5 | −218.4 | −680.2 | 66.5 | 1.9 |
| oligo55 | 69.0 | −213.0 | −656.1 | 69.0 | 1.1 |
| oligo56 | 69.0 | −185.2 | −573.5 | 69.0 | 3.4 |
| oligo57 | 69.5 | −193.9 | −601.2 | 69.5 | 4.0 |
| oligo58 | 70.0 | −185.5 | −573.9 | 70.0 | 4.1 |

| | |
|---|---|
| Mean Absolute Error | 2.4 |
| Sum of Absolute Errors | 137.9 |
| Sum of Squared Errors | 414.1 |

(*rounded to the nearest tenth)

As shown in Table 9, the Tₘs of the 58 oligonucleotides were predicted using Equation IV-1, with the mean absolute error of 2.4, the sum of absolute error of 137.9, and the sum of squared error of 414.1.

### <2-4> Tₘ Prediction Using an Equation Including the NN Parameters, the Parameters α and β

A first additional parameter α and a second additional parameter β were added to Equation I-1 to generate a modified equation for Tₘ calculation, Equation V-1.

$$Tm = \frac{Δ H ° \times 1000}{Δ S ° + α} + β ln ( length ) - 273.15 \qquad Equation V ‐ 1$$

Thirty-seven (37) reference oligonucleotides of Example <1-2> were used to determine the values of the NN parameters, the value of the parameter α, and the value of the parameter β for Equation V-1 above.

The values of the NN parameters determined were as shown in Table 10, and the values of the parameters α and β determined were −40.4 and 2.6, respectively.

TABLE 10

| | AA/ | AT/ | AG/ | AC/ | TA/ | TT/ | TG/ | TC/ | GA/ | GT/ | GG/ | GC/ | CA/ | CT/ | CG/ | CC/ |
|---|---|---|---|---|---|---|---|---|---|---|---|---|---|---|---|---|
| Pair | TT | TA | TC | TG | AT | AA | AC | AG | CT | CA | CC | CG | GT | GA | GC | GG |
| ΔS° | −23.8 | −24.7 | −23.7 | −23.3 | −25.4 | −23.8 | −23.9 | −25.5 | −25.5 | −23.3 | −21.5 | −25.9 | −23.9 | −23.7 | −24.2 | −21.5 |
| ΔH° | −8.1 | −8.3 | −8.5 | −8.4 | −7.8 | −8.1 | −8.7 | −8.8 | −8.8 | −8.4 | −8.0 | −9.4 | −8.7 | −8.5 | −9.3 | −8.0 |

(*rounded to the nearest tenth)

The Equation V-1 having the parameter values above was used to calculate the predicted Tₘs of fifty-eight (58) oligonucleotides (oligo 38-95).

The experimental Tₘ, the NN parameters, the predicted Tₘ, and the error between the predicted Tₘ and the experimental Tₘ of each of the 58 oligonucleotides (oligo 38 to 95) are shown in Table 11 below.

TABLE 11

| | Ex. | Total | Total | Predicted | \|Predicted Tₘ − |
|---|---|---|---|---|---|
| | Tₘ | ΔH° | ΔS° | Tₘ | experimental Tₘ\| |
| oligo1 | 52.5 | −191.8 | −560.0 | 54.5 | 2.0 |
| oligo2 | 53.3 | −151.2 | −432.2 | 54.4 | 1.1 |
| oligo3 | 54.0 | −159.9 | −458.7 | 55.0 | 1.0 |
| oligo4 | 55.3 | −190.3 | −551.4 | 56.7 | 1.4 |
| oligo5 | 55.8 | −173.6 | −499.1 | 56.7 | 0.9 |
| oligo6 | 56.0 | −200.1 | −583.3 | 56.1 | 0.1 |
| oligo7 | 56.5 | −224.4 | −655.2 | 58.0 | 1.5 |
| oligo8 | 56.9 | −167.9 | −480.2 | 57.3 | 0.4 |
| oligo9 | 57.0 | −161.3 | −456.6 | 59.1 | 2.1 |
| oligo10 | 57.0 | −177.2 | −510.7 | 56.3 | 0.7 |
| oligo11 | 57.5 | −201.2 | −580.6 | 59.2 | 1.7 |
| oligo12 | 58.0 | −146.5 | −411.0 | 58.9 | 0.9 |
| oligo13 | 58.0 | −154.6 | −436.9 | 58.4 | 0.4 |
| oligo14 | 58.0 | −154.6 | −437.2 | 58.2 | 0.2 |
| oligo15 | 58.0 | −183.4 | −525.8 | 58.8 | 0.8 |
| oligo16 | 58.0 | −179.3 | −512.1 | 59.4 | 1.4 |
| oligo17 | 58.0 | −154.6 | −436.9 | 58.4 | 0.4 |
| oligo18 | 58.0 | −182.0 | −522.4 | 58.3 | 0.3 |
| oligo19 | 58.3 | −152.8 | −429.6 | 59.5 | 1.2 |
| oligo20 | 58.5 | −177.8 | −507.2 | 59.6 | 1.1 |
| oligo21 | 58.5 | −185.7 | −531.5 | 59.7 | 1.2 |
| oligo22 | 59.0 | −232.7 | −678.5 | 59.3 | 0.3 |
| oligo23 | 60.0 | −155.6 | −436.2 | 60.8 | 0.8 |
| oligo24 | 60.0 | −159.7 | −452.5 | 58.5 | 1.5 |
| oligo25 | 60.0 | −144.9 | −406.1 | 58.9 | 1.1 |
| oligo26 | 60.0 | −240.7 | −699.9 | 60.8 | 0.8 |
| oligo27 | 60.5 | −153.1 | −429.9 | 60.0 | 0.5 |
| oligo28 | 60.5 | −192.8 | −551.7 | 60.6 | 0.1 |
| oligo29 | 60.5 | −226.7 | −653.7 | 62.0 | 1.5 |
| oligo30 | 60.8 | −200.8 | −575.4 | 61.3 | 0.5 |
| oligo31 | 61.4 | −199.3 | −571.7 | 60.8 | 0.6 |
| oligo32 | 61.5 | −168.1 | −474.7 | 61.1 | 0.4 |
| oligo33 | 61.5 | −257.7 | −748.5 | 62.5 | 1.0 |
| oligo34 | 61.8 | −177.3 | −506.1 | 59.3 | 2.5 |
| oligo35 | 62.0 | −161.6 | −453.6 | 61.6 | 0.4 |
| oligo36 | 62.0 | −176.0 | −500.1 | 60.4 | 1.6 |
| oligo37 | 62.5 | −188.0 | −533.3 | 62.7 | 0.2 |
| oligo38 | 62.5 | −200.2 | −574.0 | 61.1 | 1.4 |
| oligo39 | 63.0 | −169.3 | −476.5 | 62.3 | 0.7 |
| oligo40 | 63.0 | −168.9 | −474.3 | 62.9 | 0.1 |
| oligo41 | 63.0 | −234.7 | −675.1 | 63.5 | 0.5 |
| oligo42 | 63.5 | −195.7 | −556.8 | 62.7 | 0.8 |
| oligo43 | 63.5 | −177.0 | −498.1 | 63.6 | 0.1 |
| oligo44 | 63.8 | −208.5 | −598.6 | 61.6 | 2.2 |
| oligo45 | 64.0 | −171.0 | −479.8 | 63.5 | 0.5 |
| oligo46 | 64.0 | −171.1 | −481.1 | 62.7 | 1.3 |
| oligo47 | 64.0 | −177.5 | −500.3 | 63.1 | 0.9 |
| oligo48 | 64.0 | −185.2 | −521.9 | 64.3 | 0.3 |
| oligo49 | 65.0 | −195.5 | −554.7 | 63.6 | 1.4 |
| oligo50 | 65.0 | −171.1 | −479.9 | 63.5 | 1.5 |
| oligo51 | 65.5 | −185.6 | −524.1 | 63.8 | 1.7 |
| oligo52 | 65.5 | −201.3 | −572.0 | 63.9 | 1.6 |
| oligo53 | 65.5 | −178.1 | −500.7 | 63.9 | 1.6 |
| oligo54 | 66.5 | −229.5 | −648.9 | 68.4 | 1.9 |
| oligo55 | 69.0 | −222.1 | −623.5 | 69.9 | 0.9 |
| oligo56 | 69.0 | −196.8 | −548.9 | 69.0 | 0.0 |
| oligo57 | 69.5 | −205.3 | −574.5 | 69.0 | 0.5 |
| oligo58 | 70.0 | −197.0 | −549.1 | 69.3 | 0.7 |

| | |
|---|---|
| Mean Absolute Error | 1.0 |
| Sum of Absolute Errors | 55.2 |
| Sum of Squared Errors | 74.2 |

(*rounded to the nearest tenth)

As shown in Table 11, the Tₘs of the 58 oligonucleotides were predicted using Equation V-1, with the mean absolute error of 1.0, the sum of absolute error of 55.2, and the sum of squared error of 74.2.

The Tₘ prediction performances of the four (4) equations were compared. As a result, it was found that the method using Equation V-1 including both the parameters α and β in addition to the NN parameters reduced the error between the experimental Tₘ and the predicted Tₘ, thereby providing the most accurate predicted Tₘ.

Furthermore, for a sophisticated comparison between the equations for the Tₘ prediction performance, the slope of the trend line and the coefficient of determination R² were determined in a scatter plot indicating the correlation between the experimental Tₘ and the predicted Tₘ.

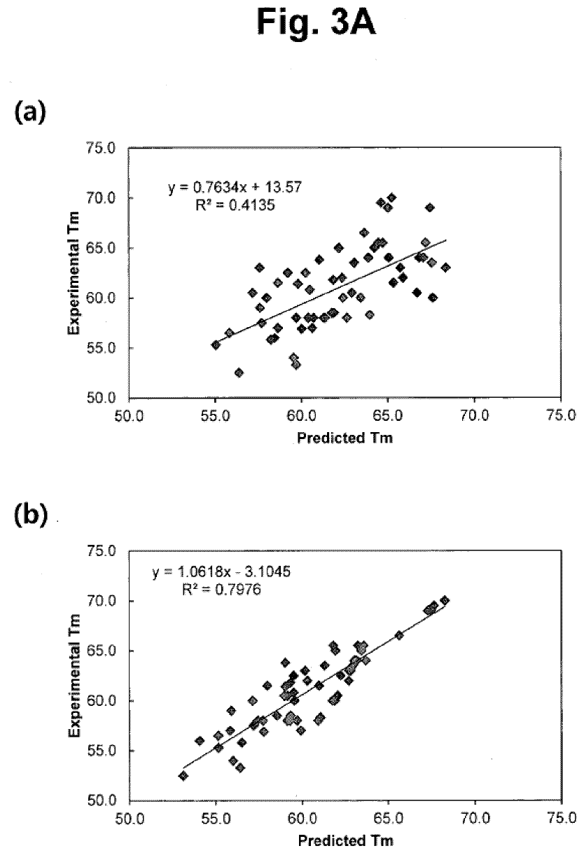

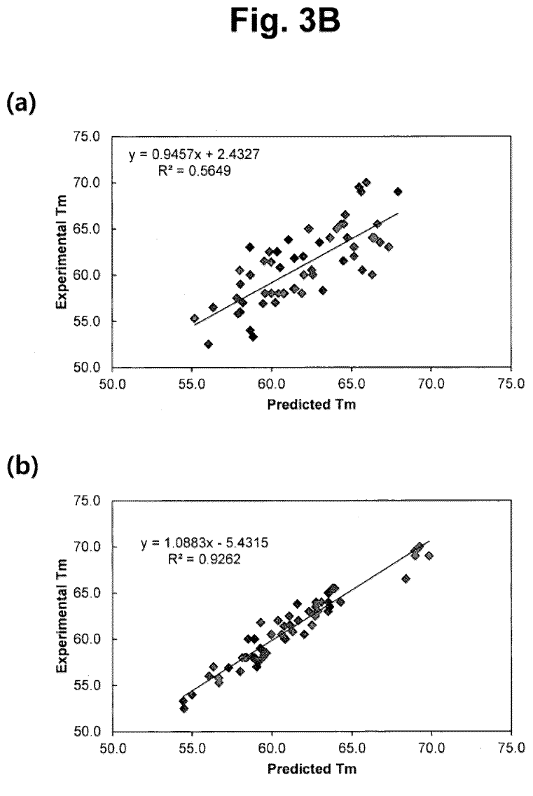

The results are shown in FIGS. 3A and 3B.

As shown in FIGS. 3A and 3B, it was found that the slope of the trend line in the scatter plot showing the correlation between the experimental Tₘ and the predicted Tₘ was 0.7634 for Equation I-1, 1.0618 for Equation III-1, 0.9457 for Equation IV-1, and 1.0883 for Equation V-1. Further, it was found that coefficient of determination R² was 0.4135 for Equation I-1, 0.7976 for Equation III-1, 0.5649 for Equation IV-1, and 0.9262 for Equation V-1.

The results demonstrate that using the equation including both the parameters α and β in addition to the NN parameters enables to obtain the predicted Tₘ most similar to the experimental Tₘ.

Having described a preferred embodiment of the present invention, it is to be understood that variants and modifications thereof falling within the spirit of the invention may become apparent to those skilled in this art, and the scope of this invention is to be determined by appended claims and their equivalents.

1-17. (canceled)

18. A method for predicting the melting temperature (Tₘ) of an oligonucleotide of interest, comprising:
(a) providing two or more reference sets, each of the two or more reference sets comprising reference oligonucleotides of varying lengths and sequences;
(b) measuring the melting temperatures (Tₘ) of the reference oligonucleotides included in each of the two or more reference sets under two or more reaction environments;
(c) establishing two or more equations for Tₘ calculation for the two or more reaction environments using (i) the sequences and (ii) Tₘ of the reference oligonucleotides included in each of the two or more reference sets; wherein the establishment of the equation comprises determining the values of nearest-neighbor (NN) thermodynamic parameters comprising the enthalpy change (ΔH) and the entropy change (ΔS), for each of NN sequences; wherein the two or more equations for Tₘ calculation have different values of the NN thermodynamic parameters for the two or more reaction environments; and
(d) predicting the Tₘ of an oligonucleotide of interest in a reaction environment in which the oligonucleotide of interest is to be placed, using an equation for Tₘ calculation established for the reaction environment among the two or more equations for Tₘ calculation.

19. The method of claim 18, wherein each of the two or more reference sets comprises reference oligonucleotides such that each of NN sequences occurs with the frequency of at least two.

20. The method of claim 18, wherein each of the two or more equations for Tₘ calculation is represented by Equation I:
$$Tm = \frac{Δ H ° \times m}{Δ S °} - n \qquad Equation I$$
wherein Tₘ is the melting temperature of the oligonucleotide; ΔH° is the sum of the enthalpy changes; ΔS° is the sum of the entropy changes; and m and n are constants.

21. The method of claim 20, wherein m is 1000 and n is 273.15.

22. The method of claim 18, wherein each of the two or more equations for Tₘ calculation comprises the parameters of the enthalpy change (ΔH) and the entropy change (ΔS) for each of nearest-neighbor (NN) sequences, and one or more additional parameters.

23. The method of claim 22, wherein the one or more additional parameters comprise a parameter for correction of entropy change and/or a parameter for correction of Tₘ contribution by length of the oligonucleotide.

24. The method of claim 18, wherein each of the two or more equations for Tₘ calculation is represented by Equation V:
$$Tm = \frac{Δ H ° \times m}{Δ S ° + α} + β ln ( length ) - n \qquad Equation V$$
wherein Tₘ is the melting temperature of the oligonucleotide; ΔH° is the sum of the enthalpy changes; ΔS° is the sum of the entropy changes; α is a first additional parameter for correction of entropy change; β is a second additional parameter for correction of Tₘ contribution by length of the oligonucleotide; length is the length of the oligonucleotide; and m and n are constants.

25. The method of claim 24, wherein m is 1000 and n is 273.15.

26. The method of claim 18, wherein the determination of the values of the parameters in step (c) is performed by linear regression or nonlinear regression.

27. The method of claim 18, wherein the determination of the values of the parameters in step (c) is performed by least square method.
